(19) United States
(12) Patent Application Publication
(10) Pub. No.: US 2004/0038201 A1
(43) Pub. Date: Feb. 26, 2004

(54) Diagnostic and therapeutic applications for biomarkers of infection

(75) Inventors: Gerard J. Nau, Sewickley, PA (US); John F. L. Richmond, Randolph, VT (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/350,797

(22) Filed: Jan. 22, 2003

Related U.S. Application Data
(60) Provisional application No. 60/350,773, filed on Jan. 22, 2002.
(60) Provisional application No. 60/356,195, filed on Feb. 12, 2002.

(51) Int. Cl. C12Q 1/70; C12Q 1/68

(52) U.S. Cl. 435/5; 435/6

(57) ABSTRACT

Distinct gene expression programs activated in response to different pathogens in macrophages are disclosed. Methods of diagnosis and treatment are also disclosed.

### RELATED APPLICATIONS

This application claims the benefit of U.S. Provisional Application No. 60/350,773, filed Jan. 22, 2002, and U.S. Provisional Application No. 60/356,195, filed Feb. 12, 2002, the entire teachings of which are incorporated herein by reference.

### BACKGROUND OF THE INVENTION

The human immune system is continually challenged by a diverse array of pathogens. The immune system first reacts to a pathogen with the innate immune response via cytokines and chemokines that modulate neutrophils, natural killer cells and other cell types, followed later by the adaptive immune response. It has been hypothesized that the innate immune system is equipped with receptors capable of recognizing many of the invading infectious agents and is able to react to optimize the immune response.

Macrophages are an important mediator of innate immunity. Macrophages are specialized cells that engulf large particles such as bacteria, yeast and dying cells by a process called phagocytosis, using opsonins (antibody, complement) or non-opsonic pattern recognition molecules, e.g., mannose receptors. Macrophages secrete signaling molecules, called cytokines and chemokines, that orchestrate the immune response, and also secrete and respond to a wide range of inflammatory mediators and play a central role in acute and chronic inflammation. In addition, macrophages can present processed foreign antigens to already primed T-lymphocytes, thereby allowing for the enhancement or inhibition of a specific immune response. Macrophages can be activated by immune stimuli to display enhanced antimicrobial resistance.

Although macrophages play a critical role in the immune response to a wide array of pathogens, little is known about the molecular components of this response. Macrophage phagocytosis of microorganisms is important in host immunity. Activated macrophages kill ingested pathogens by production of reactive oxygen and nitrogen metabolites. However, medically important pathogens, including the mycobacteria that cause tuberculosis and leprosy, evade immune destruction by surviving inside the macrophage. Survival of pathogens in macrophages is central to the pathogenesis and tissue injury in these important human diseases. An understanding of the macrophage response to pathogens is of great importance in diagnosing and treating pathogenic infection, e.g., bacterial infections such as, for example, those that result in sepsis.

### SUMMARY OF THE INVENTION

Understanding host-pathogen interactions provides insight into the pathogenesis of specific organisms and how a host responds to different organisms. Gene expression profiles of human macrophages differ depending on the organism to which the macrophages are exposed, providing clues to diagnosis. The invention described herein identifies biomarkers, biological markers of host responses, that are characteristic of infections caused by particular pathogens. Also described are biomarkers whose expression is altered in response to pathogens (common pathogen-responsive biomarkers). Described herein is a set of biomarkers that are useful as diagnostics in clinical settings. The present invention is useful in the diagnosis, prognosis and treatment of disease.

In one embodiment, the present invention is directed to a method of identifying infection by a pathogen comprising the steps of: isolating mRNA from a whole blood sample; and determining gene expression of at least one stimulus-specific gene, wherein expression of a stimulus-specific gene is indicative of infection by a pathogen to which the stimulus-specific gene is specific. In a particular embodiment, stimulus-specific gene expression is increased. In another embodiment, at least one stimulus-specific gene is not expressed. In one embodiment, the sample is an isolated monocyte sample derived from whole blood. In a particular embodiment, the isolated monocyte sample is differentiated in culture into macrophages.

For the methods described herein, the stimulus-specific gene can be selected from the group consisting of: X66867_CDS1_AT, M80899_AT, M27492_AT, X04327_AT, M34458_RNA1_S_AT, U26173_S_AT, X83492_S_AT, M68520_AT, U50062_AT, M24470_AT, M13690_S_AT, M88163_AT, L37792_AT, X02530_AT, U50527_S_AT, U00672_AT, S79639_AT, M92843_S_AT, D90070_S_AT, X83490_S_AT, U59286_AT, M87284_AT, M30818_AT, M14660_AT, L40387_AT, X99886_S_AT, HG3417-HT3600_S_AT, U50648_S_AT, M33882_AT, M13755_AT, U52513_AT, M24594_AT, D14889_AT, X57351_S_AT, U22970_RNA1_S_AT, X67325_AT, J04164_-AT, X57351_AT, X99699_AT, U10439_AT, X72755_AT, X02875_S_AT, D28915_AT, D13146_CDS1_AT, AB00015_AT, L29277_AT, L22342_AT, X63717_AT, U65416_RNA1_S_AT, M87434_AT, M62403_S_AT, U34877_AT, M59830_AT, HG4297-HT4567_AT, D86979_AT, D43949 AT, X78711_AT, U55766_AT, U56998_AT, X54489_RNA1_AT, X68277_AT, D14874_AT, M58603_AT, M31627_AT, L15326_S_AT, L09229_S_AT, L40377_AT, L00352_AT, U77180_AT, U01824_AT, M27533_S_AT, M33684_S_AT, L38608_AT, HG2981-HT3938_S_AT, U88964_AT, HG3415-HT3598_AT, M30894_AT, L08069_AT, M34455_AT, D84276_AT, U31628_AT, HG1612-HT1612_AT, M55543_AT, L31584_AT, AF005775_AT and D30755_AT. In another embodiment, the stimulus-specific gene is selected from the group consisting of: I-TAC, IL-12 p35 and IL-12 p40. The stimulus-specific gene can also be selected from the group consisting of: U75370_AT, L04270_AT, HG4334-HT4604_S_AT, X05610_AT, HG1686-HT4572_S_AT, U90902_AT, AB000584_AT, M91083_AT and L10910_AT. In a particular embodiments, the stimulus-specific gene expression is decreased. The stimulus-specific gene can be selected from the group consisting of: Z11559_AT, X62055 AT, U62293 RNA1_S_AT, S82447_S_AT, J03589_AT and D50917 AT.

In one embodiment, the present invention is directed to a method of classifying a pathogen comprising the steps of: exposing macrophages with a pathogen or immunogenic components thereof; isolating and labeling mRNA from said macrophages; detecting labeled mRNA from said macrophages such that a gene expression profile is produced; and analyzing the gene expression profile relative to one or more reference gene expression profile(s), such that similarities between the gene expression profile of the pathogen-exposed macrophage and at least one reference gene expression profile classify the pathogen as belonging to the class corresponding to the reference gene expression profile.

In one embodiment, the present invention is directed to a method of diagnosing infection in a mammal comprising the steps of: isolating mRNA from a whole blood sample obtained from the mammal; and contacting the mRNA with at least one stimulus-responsive gene probe wherein hybridization of a stimulus-responsive probe to the mRNA is indicative of infection in said mammal. In a particular embodiment, the stimulus-responsive gene probe is a stimulus-specific gene probe. In another embodiment, the stimulus-responsive gene probe is a common stimulus-responsive gene probe.

In one embodiment, the present invention is directed to a method of diagnosing infection by a pathogen in a mammal comprising the steps of: isolating mRNA from a whole blood sample obtained from the mammal; and determining gene expression of at least one stimulus-specific gene, wherein expression of the stimulus-specific gene is indicative of infection by a pathogen to which the stimulus-specific gene is specific.

In one embodiment, the present invention is directed to a method of formulating a therapeutic regimen for treating a pathogenic infection comprising the steps of: identifying a pathogen that causes the infection from a diagnostic assay performed on a whole blood sample; and formulating the therapeutic regimen according to the pathogen identified. In one embodiment, these steps can be repeated as necessary to improve the formulation of the therapeutic regimen.

In one embodiment, the present invention is directed to a method of optimizing a vaccine comprising the steps of: contacting one or more macrophages with at least one test vaccine; isolating mRNA from said macrophages; determining gene expression profiles in said macrophages; and comparing the macrophage gene expression profile to a reference gene expression profile, wherein a similarity to a pathogen-specific or pathogen-responsive gene expression profile is indicative of an optimized vaccine.

In one embodiment, the present invention is directed to an ex vivo therapeutic treatment for a disorder selected from the group consisting of pathogenic infection, sepsis and autoimmunity comprising the steps of: contacting a patient's macrophages with a pathogen or components thereof, such that the macrophages would normally signal an immune response, thereby producing activated macrophages; returning the activated macrophages to the patient such that the activated macrophages trigger an immune response against the pathogen.

In one embodiment, the present invention is directed to a method of measuring the immune response to a stimulus comprising the steps of: contacting a whole blood sample with a stimulus; isolating mRNA from the sample; and determining a gene expression profile such that the expression of at least one stimulus-responsive gene measured, thereby indicating the level of the immune response.

In one embodiment, the present invention is directed to a method of measuring the immune response to a stimulus comprising the steps of: contacting macrophages with a stimulus; isolating and labeling mRNA from the macrophages; contacting a DNA microarray with labeled mRNA from the macrophages; and measuring and analyzing the gene expression profile relative to control stimulus such that at least one stimulus-responsive gene is identified which is indicative of an immune response. In a particular embodiment, the macrophages obtained by culturing monocytes. In one embodiment, the stimulus is selected from the group consisting of bacteria, fungi, viruses, or components thereof. In one embodiment, the stimulus is selected from the group consisting of Escherichia coli, enterohemorrhagic E. coli O157:H7 (EHEC), Salmonella typhi, Salmonella typhimurium, Staphylococcus aureus, Listeria monocytogenes, M. tuberculosis, Mycobacterium bovis bacille Calmette-Guérin (BCG), lipopolysaccharide (LPS), polyI:C, and yeast mannan. In a particular embodiment, the DNA microarray is Affymetrix HU 6800. In yet another embodiment, the expression of the stimulus-responsive gene is increased in response to the stimulus. In one embodiment, the expression of the stimulus-responsive gene is decreased in response to the stimulus. In another embodiment, the stimulus-responsive gene is stimulus-specific.

In one embodiment, the present invention is directed to a method of measuring the gene expression profile in macrophages in response to a stimulus comprising the steps of: contacting immature macrophages with a stimulus; isolating and labeling mRNA from the macrophages; contacting a DNA microarray with labeled mRNA from the macrophages; and measuring and analyzing the gene expression profile relative to control stimulus such that at least one stimulus-responsive gene is identified. In a particular embodiment, the macrophages obtained by culturing monocytes. In one embodiment, the stimulus is selected from the group consisting of bacteria, fungi, viruses, or components thereof. In one embodiment, the stimulus is selected from the group consisting of Escherichia coli, enterohemorrhagic E. coli O157:H7 (EHEC), Salmonella typhi, Salmonella typhimurium, Staphylococcus aureus, Listeria monocytogenes, M. tuberculosis, Mycobacterium bovis bacille Calmette-Guérin (BCG), lipopolysaccharide (LPS), polyI:C, and yeast mannan. In a particular embodiment, the DNA microarray is Affymetrix HU 6800. In yet another embodiment, the expression of the stimulus-responsive gene is increased in response to the stimulus. In one embodiment, the expression of the stimulus-responsive gene is decreased in response to the stimulus. In another embodiment, the stimulus-responsive gene is stimulus-specific.

In one embodiment, the present invention is directed to a method for generating a database of stimulus-responsive genes comprising the steps of: contacting macrophages with a stimulus; isolating and labeling mRNA from the macrophages; contacting a DNA microarray with labeled mRNA from the macrophages; and measuring and analyzing the gene expression profile relative to a control stimulus such that a database comprising at least one stimulus-responsive gene is generated.

In one embodiment, the present invention is directed to a method of generating a database of stimulus-specific genes comprising the steps of: contacting immature macrophages with a stimulus; isolating and labeling mRNA from the macrophages; contacting a DNA microarray with labeled mRNA from the macrophages; and measuring and analyzing the gene expression profile relative to control stimulus such that a database of stimulus-specific genes comprising at least one stimulus-specific gene is generated.

In one embodiment, the present invention is directed to a method of generating a database of common stimulus-responsive genes comprising the steps of: contacting macrophages with a stimulus; isolating and labeling mRNA from the macrophages; contacting a DNA microarray with labeled mRNA from the macrophages; and measuring and analyzing the gene expression profile relative to control stimulus such that a database of common stimulus-responsive genes comprising at least one common stimulus-responsive gene is generated

In one embodiment, the present invention is directed to a database of common stimulus-responsive genes. In a particular embodiment, the database can comprise one or more genes selected from the group consisting of: GCSF, GMCSF, IL12B, IL1RN, IL6, IL6, PBEP, ProI1B, TNFA, IL8, IL8, IP10, MCP1, MGSA, MIP1A, MIP1B, MIP2A, MIP2B, RANTES, CD44, CD44, ICAM1, IFITM1, LAMB3, NINJ1, TNFA1P6, ADORA2A, CCR6, CCR7, CCRL2, DTR, EBI3, HM74, ILI5RA, IL7R, LDLR, P2RX7, P2XR, PLAUR, PVR, SLAM, TNFRSF5, TXN, CNK, DUSP1, DUSP2, DUSP5, EBI2, GBP1, HCK, INHBA, JAG1, KYNU, LIMK2, MAP2K3, MAP3K4, MINOR, NAF1, NFKB1, PDE4B, PPP3CC, PTPN1, TRAF1, TRIP10, DSCR1, ELF4, ETS2, IRF1, IRLB, JUNB, MRF-1, NFKBIA, NFKBIE, NFKBp50, STAT4, STAT5A, TSC22, XBP1, ZFP36, COX2, COX2, COX2, GCH1, PTX3, BIRC2, BIRC3, BIRC3, CFLAR, IER3, TNFAIP3, BTG1, BTG3, TNFRSF9, TNFSF9, DAP, MMP1, MMP10, MMP14, SERPINB2, SERPINB8, GADD45A, HSPA1A, SOD2, SOD2, ATP2B1, NRAMP2, SLC7A5, ADA, AMPD3, beta-1,4-galatosyl transferase, BF, CKB, GJB2, GLCLR, HSD11B1, INDO, MTF1, ADM, ARHH, B4-2, BRCA2, CD83, GEM, GOS, GYPC, H2AFO, HIVEP2, ISG15, ISG20, MACMARKS, MIG2, MXJ, RCN1, SDC4, SNL, TNFAIP2, TSSC3, K1AA0105, KIAA0172, GCHFR, CAT, LTA4H, CD14, ALOX5, MCP1, PECAM1, SPARC, RARA, TNFRSF1A, ENG, L77730, CD36L1, CD163, TGFBR2, GSF1R, MRC1, CD32, P2RX1, SRPK2, SLA, MERTK, DAB2, SF3A3, EGR2, NFATC3, MX11, FOS, Hbrm, SLC29A1, UPA, FGL2, TBXAS1, SGSH, PPP2R5C, IDH2, LPL, MPI, PYGL, HSD17B4, RNASE6, GALC, GLCLC, RNASE1, ME1, PURA, MYO1E, VCL, IV12B, ADFP, MNDA, STAB1, TGFBI, KIAA0022, AD000092, D87075, U79288, P311, HG2090-HT2090, HG2090-HT2090 and HG2090-HT2090.

In one embodiment, the present invention is directed to a method of identifying a pathogen comprising the steps of: contacting one or more immature macrophages with a stimulus; isolating and labeling mRNA from said macrophages; contacting a DNA microarray with labeled mRNA from said macrophages; and measuring and analyzing the gene expression profile relative to control stimulus such that at least one stimulus-specific gene is identified thereby identifying a pathogen for which the stimulus-specific gene is specific.

In one embodiment, the present invention is directed to a method of diagnosing infection by a pathogen comprising the steps of: isolating mRNA from a whole blood sample; and determining a gene expression profile such that at least one stimulus-specific gene is identified, thereby identifying the pathogen.

In one embodiment, the present invention is directed to a method of diagnosing infection by a pathogen comprising the steps of: isolating and labeling mRNA from at least one whole blood sample from a mammal; contacting a DNA microarray with labeled mRNA from the sample; and measuring and analyzing the gene expression profile relative to control stimulus such that at least one stimulus-specific gene is identified thereby identifying the pathogen for which the stimulus-specific gene is specific.

In one embodiment, the present invention is directed to a method of diagnosing infection in a mammal comprising the steps of: isolating proteins from one or more samples from the mammal; and contacting the proteins with at least one stimulus-specific antibody, wherein binding of the stimulus-specific antibody to one or more proteins is indicative of infection in the mammal.

In one embodiment, the present invention is directed to a method for screening for an agent that induces a pathogen-specific immune response comprising the steps of: contacting at least one macrophage with a test agent; isolating mRNA from the macrophage(s); determining a gene expression profile from the isolate mRNA; and comparing the gene expression profile obtained from the macrophages contacted by the test agent with a reference gene expression profile of an activated macrophage, wherein a similarity in gene expression profiles indicates the test agent induces a pathogen-specific immune response.

In another embodiment, the invention is directed to a method of identifying a genus-specific response comprising the steps of: isolating mRNA from a whole blood sample obtained from a mammal; contacting a DNA microarray with labeled mRNA from said macrophages; and comparing the gene expression profile to a reference genus-specific-induced gene expression profile, wherein a similarity indicates a genus-specific response. In a particular embodiment, the reference gene expression profile comprises expression values for IFN-β.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A and 1B depict macrophage activation program elicited in response to a variety of bacteria and bacterial components. A) The macrophage activation program. Macrophage gene expression was measured at 1, 2, 6, 12 and 24 hours after introduction of bacteria or latex beads. One hundred and ninety-eight genes met criteria for significant changes upon exposure of six of eight bacteria studied. The genes are ordered along the y-axis by category, with the degree of change indicated by greyscale intensity along the bar. B) A subset of bacterial components elicit much of the activation program (induced genes shown only). Control experiments demonstrated that residual endotoxin contamination of the recombinant hsps was not responsible for the extent of cytokine expression induced by hsps (FIGS. 8A-C).

FIGS. 2A and 2B are graphical displays showing differences in expression profiles that distinguish between bacteria. A) Differential gene expression in macrophages exposed to M. tuberculosis, E. coli, or S. aureus. The difference index represents how expression levels induced by the three bacteria differ from the average expression level induced by all bacteria studied: large values are assigned to genes whose expression levels exhibit the greatest differences in specific bacterial infections. Multiple time course experiments were used (M. tuberculosis: 2 repeats, E. coli: 3 repeats, S. aureus: 2 repeats). For every gene within each profile, the responses of macrophages to each of the bacterial species in question were compared to the average response to all bacterial species studied. Statistically significant differences were identified using Student's t-test (p<0.005). The difference index was calculated as follows:

D.I.=|log₂(fold-change of gene X in infection A)−log₂(avg fold change of gene X in all infections)

B) M. tuberculosis induced lower levels of macrophage IL-12 and IL-15 gene expression than the average expression measured across all data sets. The average fold-change values observed after exposure to M. tuberculosis (▴) are displayed for the time course. In comparison, the average fold-change values across all data sets was significantly greater (▪).

FIGS. 3A and 3B are graphical representations showing M. tuberculosis-induced repression of IL-12 protein production. M. tuberculosis induced less IL-12 protein than E. coli and suppressed IL-12 production induced by E. coli. IL-12 p40 accumulation in supernatants of macrophages 12 hours after exposure to either M. tuberculosis, E. coli, or both bacteria was measured by ELISA (R&D Systems, Minneapolis, Minn.). Additional experiments demonstrated this response was reproducible and not donor dependent. TNF-α ELISA was performed on supernatants from the same cultures after 4 hours (peak expression of this cytokine preceding the wash step). Values are means+/−standard deviations of replicate ELISA measurements. Neither IL-12 nor TNF-α were detected in macrophages treated with media alone.

FIGS. 4A-4C are graphical representations showing human macrophage gene expression changes in response to bacteria. A) Macrophages exhibit a common gene expression response to a variety of bacteria. A hierarchical clustering of the 977 genes whose changes in expression were considered significant upon exposure to at least one of the eight bacteria tested. The data are displayed in an x, y, z plot highlighting the magnitude of the gene expression changes along the vertical (y) axis. Genes are ordered along the z-axis by a hierarchical clustering algorithm (Eisen, M. et al., 1998. Proc. Natl. Acad. Sci. USA, 95:14863-14868), and the time course along the x-axis. The display was generated using the MatLab program (MathWorks, Natick, Mass.). B) The individual 24 hour time courses in the x-axis illustrate the minimal changes of gene expression after the introduction of latex beads compared to bacteria. C) View along the z-axis, showing the expression changes of the 977 genes.

FIGS. 5A and 5B are representations showing the identified genes that are constitutively expressed in macrophages. A) Gene expression of unstimulated human fibroblasts, both primary and 4 cell lines (TIP5neo, TIP5 neo hygro, TIP5 hTERT and TIP5 hTERT hygro), was monitored as described (Lockhart, D. et al., 1996. Nat. Biotechnol., 14:1675-1680) using Affymetrix Hu6800 GeneChips™. For each gene, the average of the log-transformed fluorescence intensities of the 5 fibroblast datasets was calculated. A similar calculation was performed for the 10 unstimulated macrophage datasets. Statistically significant differences were identified using Student's t-test (p<0.001); those genes are shown here. The genes are ordered by magnitude of the macrophage/fibroblast ratios (average fluorescence intensity). Ratios of greater than 1 indicate that a gene is preferentially expressed in macrophages, while ratios less than 1 indicate that a gene is more highly expressed in fibroblasts. B) Cathepsin genes, involved in antigen processing and presentation, were not identified in the common signature of macrophage genes induced after exposure to bacteria. Expression levels of these genes were higher in the macrophages than in fibroblasts. The cathepsin genes are examples of macrophage genes that are constitutively expressed and that are not altered by exposure to bacteria.

FIGS. 6A and 6B are representations showing controls for the macrophage time course experiment. A) Reproducibility of macrophage gene expression measurements. RNA from duplicate macrophage cultures was harvested and gene expression was measured using Affymetrix HU6800 GeneChips™. Relative fold-change values were determined for each gene by dividing the fluorescence intensities of the two samples. Results are displayed for those genes whose expression was called ‘present’ (detected) by the Affymetrix algorithm. B) Culturing macrophages over a 24 hour time course results in small changes in gene expression. Macrophages were cultured in media (DMEM, 1% human serum, gentamicin) over a 24-hour time course. Gene expression levels (fluorescence intensities) in the time courses were compared to time zero controls to calculate fold-change. The fold-change expression levels were clustered by pattern of expression in the two repeated time courses shown above. A total of 126 and 100 genes in time courses 1 and 2, respectively, were found to increase or decrease expression levels greater than 2-fold in 2 consecutive time points.

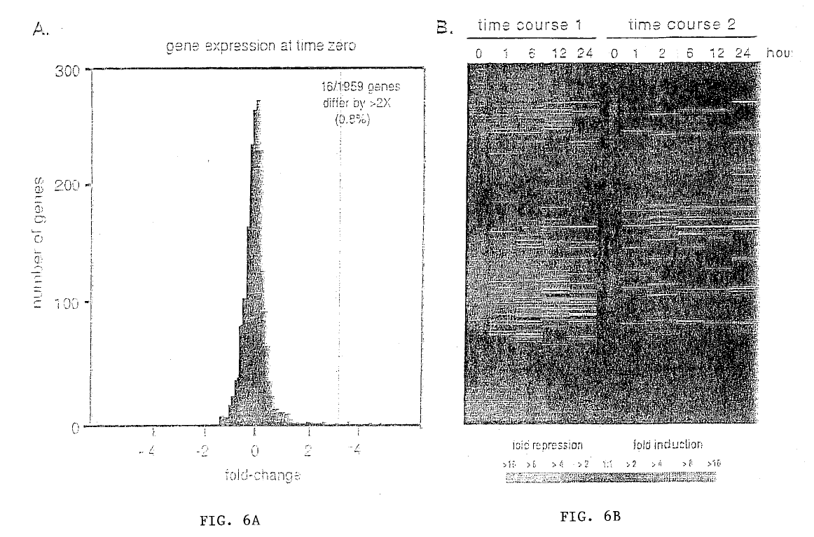

FIG. 7 is a representation showing experiment- or donor-specific macrophage gene expression. Donor-specific changes in gene expression were identified using two analyses: 1) significant gene expression change in all treatments of only one donor, but not in any other; 2) expression, averaged over each treatment, in one donor was either 2-fold higher or 4-fold higher than averaged expression in the other three donors. Genes that met these criteria were hierarchically clustered and are shown above.

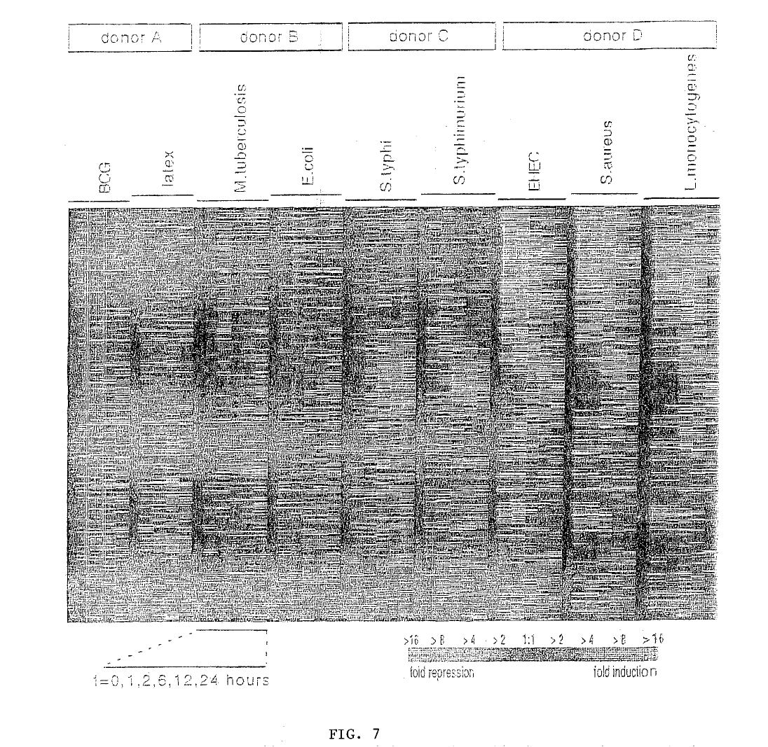

FIGS. 8A-8C are representations of data showing that endotoxin contamination is not responsible for hsp-induced cytokine secretion. A) SDS-PAGE analysis of recombinant TBhsp71 and BCG hsp65 (Stressgen) demonstrates the purity of these preparations. Increasing μg loaded left to right. The first 3 samples were run under reducing conditions, while the next three were run without reduction. B). Titrations of LPS demonstrate the sensitivity of macrophages to endotoxin. Roughly 100-fold more endotoxin was required to elicit IL-12 p40 than TNF-alpha, and endotoxin levels lower than 1.4 Eu/mL did not stimulate production of either cytokine. Cytokines were quantitated by ELISA. C) Endotoxin-equivalent amounts of hsp and LPS (0.06 Eu/mL=13 pg/mL LPS, 10 μg/mL hsp 70) demonstrate that contaminating levels of endotoxins are not responsible for the extent of cytokine production by hsps. Hsp70 elicited 3-6 times more cytokine than the endotoxin-equivalent amount of LPS. Cytokines were quantitated by ELISA.

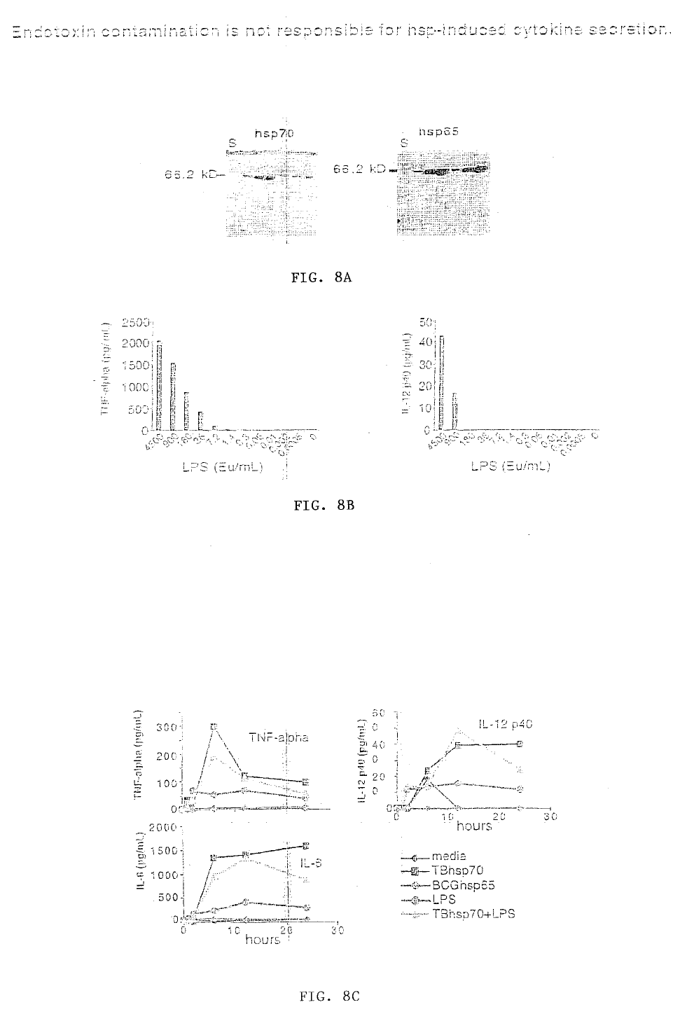

FIG. 9 is a graphical representation showing a specific TLR-4 antagonist inhibits production of TNF-alpha by hsp70. Monocytes were plated at 20×10⁵ cells/well in 96-well Primaria plates and matured to macrophages as described. Cells were washed with Hank's Balanced Salt Solution incubated overnight in DMEM, 1% human serum, and pretreated for 30 minutes at 37° C. with 1 μg/mL Rhodobacter sphaeroides lipid A (RSLA). RSLA is a specific antagonist of TLR-4. Cells were then stimulated with 0.1 MOI E. coli sd-4, 1 μg/mL LPS, or 10 μg/mL hsp70 to duplicate, and culture media was collected at 0, 2, 6, and 12 hours. ELISAs were performed using manufacturer's protocols. Results are displayed as percent TNF-alpha level observed in the absence of RSLA pre-treatment.

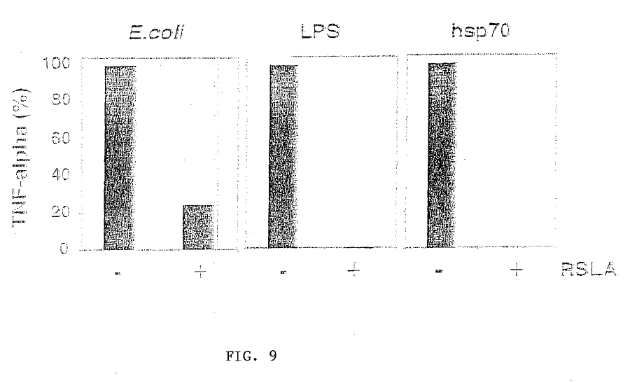

FIG. 10 is a graphical representation showing that E. coli MOI has a small but detectable effect on the macrophage gene expression signature. Macrophages were exposed to E. coli sd-4 at MOIs of 0.05:1, 0.5:1, 5:1, and 50:1 over a 24-hour time course. Macrophage RNA was harvested and gene expression was analyzed. MOIs were confirmed by culturing serial dilutions of E. coli on LB/agar plates and counting colonies.

FIGS. 11A and 11B are graphical representations of gene expression in macrophages exposed to pathogens. A) Gene expression changes in macrophages exposed to media, Gram-negative, or Gram-positive bacteria. Fold change is expression at various times after introduction of bacteria divided by expression of untreated macrophages at time=0. Average log fold change of nine (Gram-negative) and four (Gram-positive) time courses are shown with one representative media time course. Insert shows side view. B) Gene expression changes in macrophages exposed to media ♦, Gram-negative ●, or Gram-positive ▴ bacteria of three genes from FIG. 11A.

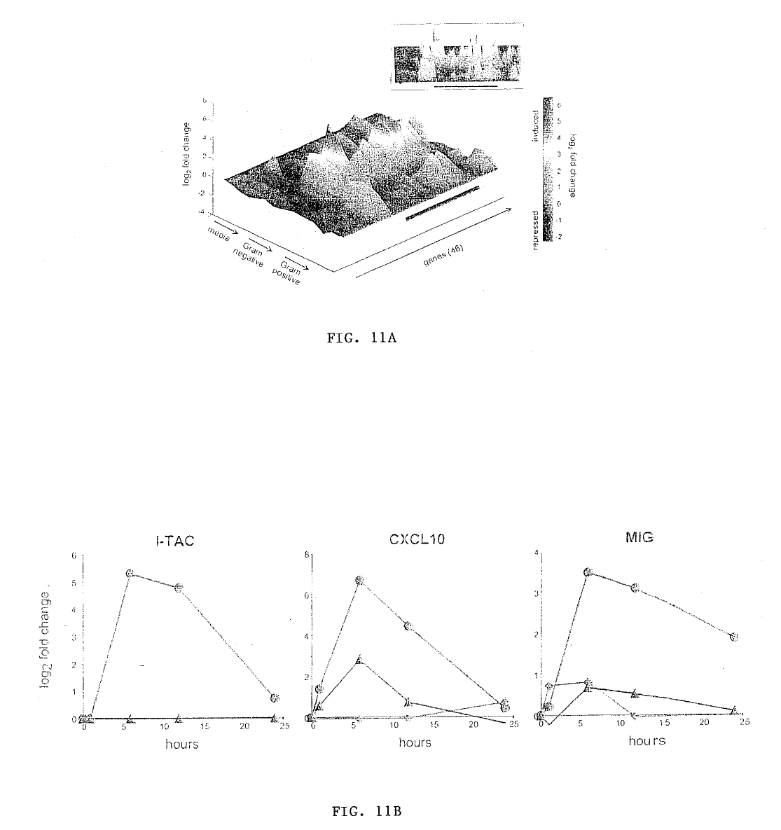

FIGS. 12A-12C are graphical representations showing pathogen effects on macrophages. A) Cytokine accumulation in supernatants of macrophages treated with various stimuli. Supernatants were harvested 24 hours after introduction of bacteria. I-TAC was only induced by Gram-negative bacteria (left panel). The macrophages were activated by all bacteria as illustrated by IL-12 p40 production (right panel). Values are mean±standard deviation of replicate measurements within the ELISA. Similar results were observed in multiple experiments. B) I-TAC production by macrophages and dendritic cells. Supernatants were harvested 24 hours after exposure to Gram-negative LPS or Gram-positive lipoteichoic acid. I-TAC concentrations were normalized to maximum level of secretion for each cell type. (macrophages=2.8 ng/mL, dendritic cells=59 ng/mL). C) Association of IFN-β from macrophages exposed to Escherichia. Average gene expression changes of IFN-β in macrophages exposed to E. coli (lab strain sd-4 and O157:H7, 5 time courses) and S. typhi and S. typhimurium (2 time courses each; left panel). Supernatants were harvested (right panel) 4 hours after introduction of bacteria and were tested for IFN-β protein by commercial ELISA (Biosource International, Inc.).

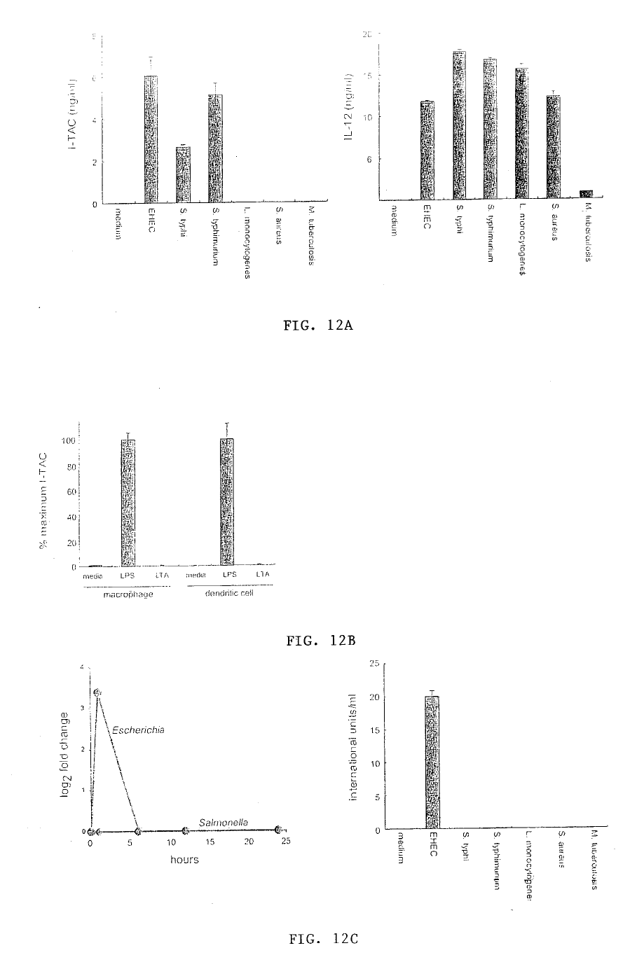

FIGS. 13A and 13B are graphical representations showing class-specific differences in macrophage gene expression induced by bacteria and purified bacterial components. A) Three-dimensional plot of gene expression changes in macrophages exposed to media, Gram-negative, or Gram-positive bacteria. Average expression changes of time course data shown for macrophages exposed to Gram-negative bacteria (nine time courses), Gram-positive bacteria (four time courses), or media (four time courses). Hierarchical clustering identified 43 genes induced most robustly by Gram-negative bacteria (pink bar). They were clustered using previously described algorithms (see FIG. 4) B) TLR-specific bacterial components recapitulate the differential expression patterns. Expression changes from A) represented in 2-dimensional format with the TLR4-induced core cluster marked by the pink bar (left). Expression changes in macrophages exposed to TLR4 agonist LPS compared to non-TLR4 agonists LTA, MDP, and fMLP (right).

FIG. 14 is a set of graphical matrices showing genes in the core cluster induced by TLR4 agonists and regulation by IFNs. A core cluster of 43 genes is most different between macrophages stimulated with Gram-negative and Gram-positive bacteria (left panel). NCBI/LocusLink-compatible gene names are on left. Those genes regulated by IFNs, based on PubMed search of gene name, are identified by an asterisk (*). Gene expression changes in macrophages are induced by IFNs but not by IL-10 or IL-12 (right panel). The averaged response to Gram-negative bacteria is included for comparison. All IFNs were used at 1000 units/mL. The color scale depicting the magnitude of expression change is the same as that used in FIG. 13B.

FIGS. 15A-15C are graphical representations showing the correlation between I-TAC gene expression and protein production with exposure to Gram-negative bacteria, LPS, and IFN. A) Line graph of 1-TAC gene expression derived from microarray data. Average log₂-fold change of 1-TAC expression induced by Gram-negative (●) and by Gram-positive (▴) bacteria are shown (left panel). Comparable levels of IL-12 p40 expression were induced by both classes of bacteria (right panel). B) Protein levels of 1-TAC and IL-12 p40 induced by different bacteria. Gram-negative bacteria, but not Gram-positive bacteria, induce protein production of 1-TAC (left panel). Means±SD of replicate measurements within the ELISA are shown. Supernatants were derived from macrophage cultures in 5 mL media at 10⁷ cells/25 cm² as performed previously (Nau, G. et al., 2002. Proc. Natl. Acad. Sci. USA, 99:1503-1508). Comparable levels of IL-12 p40 were produced by both classes of bacteria (right panel). Class-specific induction of 1-TAC was observed in more than 5 experiments with different donors. C) I-TAC production in response to TLR4-specific LPS but not other TLR agonists. E. coli LPS (1 μg/mL) induces I-TAC production but synthetic MDP (100 μg/mL) does not (left panel). Results of mean±SD of replicate measurements within the ELISA are shown. Similar patterns of 1-TAC production were seen with Salmonella LPS and LTA in other experiments. Both LPS and MDP induce IL-12 p40 production (right panel).

FIGS. 16A and 16B are graphical representations demonstrating that Type I IFN is sufficient and necessary for the production of 1-TAC by macrophages stimulated with E. coli. A) I-TAC production in response to different IFNs. Macrophages in microtiter wells (2×10⁴/well) were stimulated with 100 units/mL IFN. Results are mean±SD of replicate wells in the experiment. Similar patterns of 1-TAC production were seen in 3 experiments with different donors; in 2 additional experiments IFN-β and IFN-γ induced comparable levels of I-TAC. B) A monoclonal antibody that binds the second chain of Type I IFN receptor (IFNAR2) blocks I-TAC production by macrophages stimulated by E. coli. An isotype control antibody fails to inhibit I-TAC production (left panel). The IFNAR2 antibody does not interfere with IL-12 p40 production (right panel). Data are mean±SD of replicate microtiter wells (2×10⁴ cells/well) within an experiment. Similar results were seen in 4 donors.

FIGS. 17A-17C are graphical representations showing the regulation of IL-12 p70 production by human macrophages. A) More p70 was produced by macrophages exposed to Gram-negative bacteria. The same supernatants tested for I-TAC and IL-12 p40 displayed in FIG. 15B were used. Similar results were seen in 3 donors. B) p70 is produced by macrophages stimulated with LPS but not by MDP. The same supernatants tested in FIG. 15C were used here. Similar results were seen in two other donors that were tested. C) Anti-IFNAR2 fails to inhibit IL-12 p70 production. The same supernatants tested in FIG. 16B were used in the HS ELISA. Similar results were seen in 3 donors that were tested. All results in this figure represent mean±SD of replicates within the HS ELISA.

FIG. 18 is a graphical representation showing IL-12 p70 is produced by whole blood cells exposed to E. coli but not S. aureus. Washed, whole blood was diluted 1:2 with 10% human serum/DMEM and incubated with 5×10⁷ bacteria/mL of blood for 24 hours. Data are mean±SD of replicates within the DuoSet ELISA. Similar responses to TLR4 stimulation were seen in 5 experiments using 3 donors.

### DETAILED DESCRIPTION OF THE INVENTION

Studying host-pathogen interactions using a parallel, comparative analysis of gene expression permitted the identification of both shared and distinct gene expression responses in the macrophages. The shared macrophage activation program is robust and is induced by gram-positive bacteria, gram-negative bacteria, and mycobacteria. The pro-inflammatory component of the activation program may constitute a generic ‘alarm signal’ that marshals anti-bacterial defenses. However, the majority of the activation program is comprised of cell surface proteins and signaling molecules engendering new functions in the macrophage, suggesting a maturation process similar to that observed with dendritic cells (Hamerman, J. and Aderem, A., 2001. J. Immunol., 167:2227-2233).

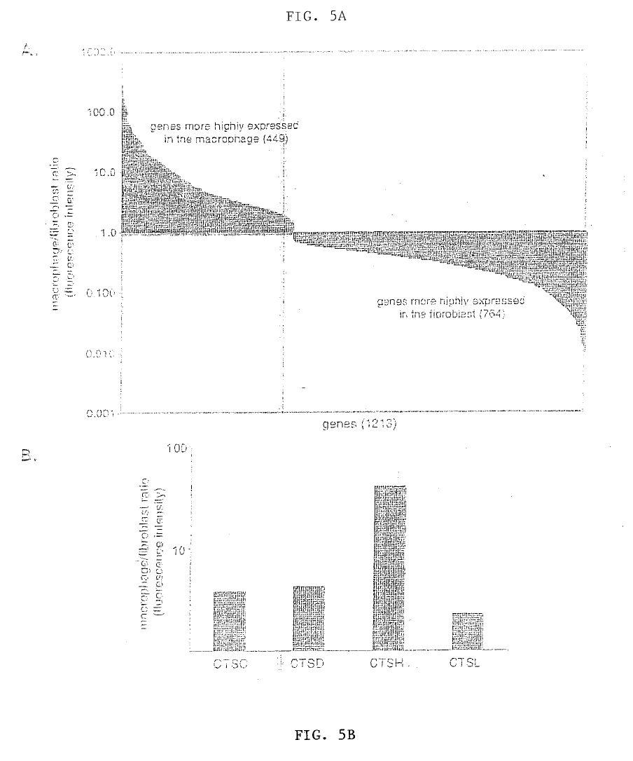

A subset of bacterial components are able to induce the activation program. These components are all agonists for Toll-like Receptors (TLRs): LPS for TLR4 (Chow, J. et al., 1999. J. Biol. Chem., 274:10689-10692), LTA and MDP for TLR-2, and heat shock proteins for TLR2 and TLR 4 (Vabulas, R. et al., 20001. J. Biol. Chem., 276:31332-31339; Ohashi, K. et al., 2000. J. Immunol., 164:558-561). The ability of hsps to elicit this response is likely to account for their potency as adjuvants in pre-clinical (Chu, N. et al., 2000. Cell Stress Chaperones, 5:401-405) and clinical vaccine trials (Janetzki, S. et al., 2000. Int. J. Cancer, 88:232-238). In contrast, the other components tested are not known to activate TLR. Notably absent from the activation program were several categories of genes commonly associated with macrophage functions, such as genes involved in antigen presentation. However, as described herein, such genes are highly expressed in macrophages in the absence of bacterial stimulation. Comparison of gene expression profiles of unstimulated human macrophages to unstimulated human fibroblasts revealed that cathepsins, HLA-D genes, and others relevant to macrophage functions were preferentially expressed in the macrophages (FIGS. 5A and 5B).

This study of host-pathogen interactions has provided insights into host defenses and pathogen-specific manipulations of those defenses that have practical applications. Understanding the macrophage activation program, and the bacterial components that elicit it, will be useful in designing vaccines and cytokine therapies that engage the innate immune system in a targeted fashion. These expression data described here should provide a foundation for further studying the pathogenesis of these and other infectious agents such as fungi, viruses, and parasites.

The present invention is based not only in a shared macrophage activation program, but also, at least in part, on the discovery that exposure of macrophages to a pathogen results in a pathogen-specific pattern of gene expression. Thus, the present invention can be used to identify infectious agents, e.g., gram-negative bacteria, gram-positive bacteria, mycobacteria, and pathogenic components of such bacteria, based on gene expression profiles described herein. The present invention also provides methods for determining pathogen-specific pattern of gene expression for additional pathogens or components thereof. The findings described herein identify the key component(s) of each pathogen that triggers an immune response, thus providing robust targets for immunotherapy. Therefore, the present invention provides a better understanding of host-pathogen interaction, aids in identification of known and novel pathogens, and improves the diagnosis and treatment of infectious diseases.

Host-pathogen relationships are characterized by the complex interplay between host defense mechanisms and attempts to circumvent these defenses by microorganisms (Knodler, L. et al., 2001. Nat. Rev. Mol. Cell Biol., 2:578-588; Fortunato, E. et al., 2000. Trends Microbiol., 8:111-119). Macrophages play key roles in host defense by recognizing, engulfing and killing microorganisms. Among the microorganisms recognized by macrophages, bacteria are an important and highly diverse class of human pathogens. Bacterial pathogens that overcome host defenses ensure their ability to survive and propagate (Pieters, J., 2001. Curr. Opin. Immunol., 13:37-44). Therefore, a thorough understanding of the normal host response to bacteria provides a foundation to understand bacterial tactics for evading these responses and thus disease prevention.

The extent to which macrophage responses to different bacteria are distinct or similar at the level of transcription is not understood well. Given the cellular components shared between bacteria and the signaling pathway shared by the Toll-like receptors (TLR) that respond to these components (Akira, S. et al., 2001. Nat. Immunol., 2:675-680), it is possible that macrophages respond to all bacteria in a standard fashion. However, the diversity of bacteria and the differences in their pathogenesis suggests pathogen-specific responses. For example, some microorganisms, like Mycobacterium tuberculosis, survive within macrophages and could be expected to elicit specific changes in their host phagocytes.

The parallel, comparative analysis of gene expression revealed clues to M. tuberculosis pathogenesis. This organism induced little IL-12 and IL-15 relative to other bacteria, and repressed IL-12 production. Such a pathogen-specific response suggests that signaling pathways other than those mediated by TLR are active, leading to distinctive changes in gene expression. The poor induction of IL-12 by M. tuberculosis is consistent with observations of macrophage responses to M. tuberculosis and to recombinant M. smegmatis transformed with the 19 kDa antigen of M. tuberculosis (Giacomini, E. et al., 2001. J. Immunol., 166:7033-7041; Post, F. et al., 2001. Infect. Immun., 69:1433-1439). The effect of M. tuberculosis on macrophage IL-12 production appears similar to the inhibition of IL-12 production observed with Leishmania major and Histoplasma capsulatum, two other intracellular pathogens (Carrera, L. et al., 1996. J. Exp. Med., 183:515-526; Marth, T. and Kelsall, B., 1997. J. Exp. Med., 185:1987-1995). This may represent convergent evolution of organisms selected for their ability to survive within macrophages. IL-12 plays a fundamental role in generating Th1 immune responses (Magram, J. et al., 1996. Immunity, 4:471-481) and is critical for host resistance to tuberculosis infection in mice and in humans (Cooper, A. et al., 1997. J. Exp. Med., 186:39-45; Flynn, J. et al., 1995. J. Immunol., 155:2515-2524; Altare, F. et al., 1998. Science, 280:1432-1435; de Jong, R. et al., 1998. Science, 280:1435-1438; Altare, F. et al., 1998. J. Clin. Invest., 102:2035-2040). Repression of 1L-12 production may enhance the survival of M. tuberculosis against the innate immune response or the developing adaptive immune response. This notion is supported by two observations. First, supplemental IL-12 enhances the ability of normal mice to clear a mycobacterial infection (Doherty, T. and Sher, A., 1998. J. Immunol., 160:5428-5435). Second, exogenous IL-12 therapy has rescued two patients, one with a pulmonary M. abscessus infection and one with disseminated M. tuberculosis infection, whose infections were refractory to antibiotics and supplemental IFN-γ (Holland, S., 2000. Adv. Intern. Med., 45:431-452; Greinert, U. et al., 2001. Eur. Respir. J, 17:1049-1051). The findings described herein support the idea that both IL-12 and IL-15 could be useful therapies for clinical tuberculosis suggested by the animal models (Maeurer, M. et al., 2000. Infect. Immun., 68:2962-2970).

Disclosed herein is a detailed, comparative examination of the transcriptional responses of macrophages to a variety of bacteria, including a number of pathogens. This experimental approach allowed for the discovery of the themes that define the innate immune responses of macrophages to pathogens. The macrophage transcriptional responses also discriminate between different pathogens, and analysis of the response to M. tuberculosis yielded insights into the mechanism of pathogenesis.

Identification of pathogens that cause disease is of paramount importance in providing effective treatment, as treatments can vary widely depending on the pathogen. In one embodiment, the present invention is a method of identifying a pathogen that has infected a vertebrate (e.g., a mammal, such as, for example, a human) comprising the steps of isolating mRNA from one or more macrophages from the vertebrate; and determining a gene expression profile of at least one stimulus-specific gene, wherein the gene expression profile is indicative of infection by a pathogen to which the stimulus-specific gene(s) is specific. Methods of isolating RNA are described herein and well known to one of skill in the art. “Gene profile” or “gene expression profile” as used herein are defined as the level or amount of gene expression of one or more particular genes as assessed by methods described herein or other methods known in the art. The gene expression profile can comprise data on one or more genes and can be measured at a single time point or over a period of time.

Pathogens can be identified by comparing the gene expression profile obtained from macrophages from a vertebrate exposed to the pathogen with one or more stimulus-specific gene expression profiles (e.g., in a database). The pathogen can optionally be a family or class of pathogens that is identified by a particular gene expression profile. For example, the pathogen can be a member of the Eschericia family (e.g., E. coli), a member of the Candida family (e.g., Candida albicans) or a mycobacterium. Pathogens can be classified as gram-negative bacteria (Escherichia coli, enterohemorrhagic E. coli O157:H7 (EHEC), Salmonella typhi, and Salmonella typhimurium), gram-positive bacteria (Staphylococcus aureus and Listeria monocytogenes), or mycobacteria (M. tuberculosis and Mycobacterium bovis bacille Calmette-Guérin (BCG)). Therefore, the present invention can identify the class, genus or species of pathogen causing a disease or illness and thus, aid in the selection of a treatment regime. A “treatment regime” as used herein refers to the clinical therapy a patient receives to ablate, alleviate or attenuate the disease or disorder. Additionally, the present invention allows for the identification of a particular pathogen, e.g., E. coli, Candida albicans, EHEC, Salmonella typhi, Salmonella typhimurium, Staphylococcus aureus, Listeria monocytogenes, M. tuberculosis or BCG.

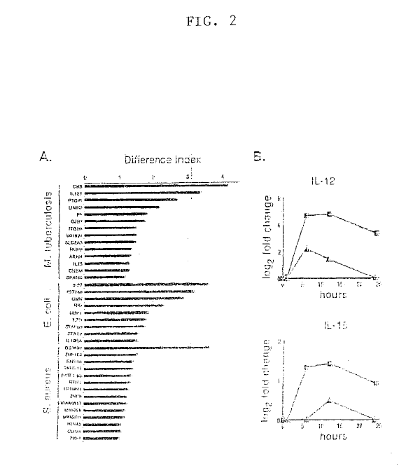

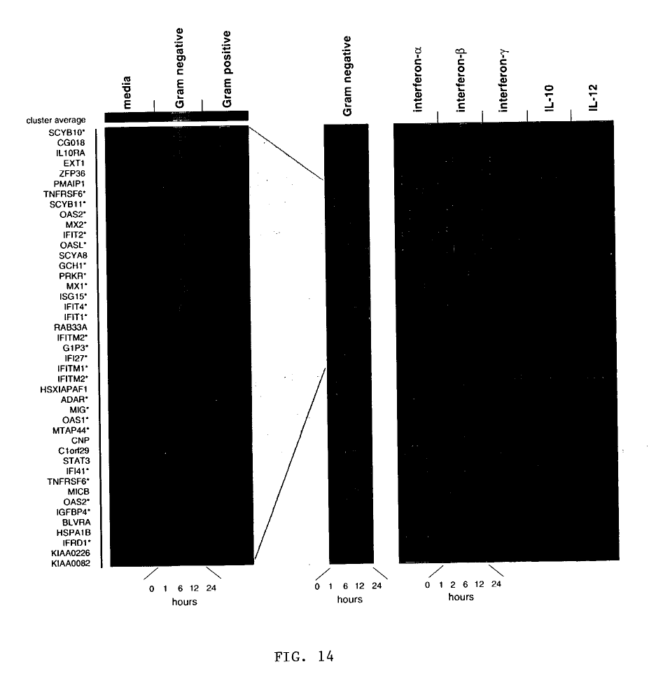

Additionally, the present invention provides stimulus-responsive, stimulus-specific and common stimulus-responsive (Table 1, FIGS. 2 and 14) genes. Thus, the present invention provides information regarding the genes that are important in the selective immune response to a specific pathogen and in the common immune response elicited by all infecting organisms, thereby providing additional targets for diagnosis and therapy.

“Stimulus-responsive” genes, as used herein, refers to genes that are regulated in response to a pathogen or a component of a pathogen that elicits an immune response. “Stimulus-responsive” and “pathogen-regulated” can be used interchangeably. “Stimulus-specific” genes, as used herein, refers to genes that are specifically-regulated by a pathogen, pathogen-class or component thereof. “Stimulus-specific” and “pathogen-specific” can be used interchangeably. “Common stimulus-responsive genes”, as used herein, refers to genes that exhibit altered expression in response to two or more pathogens, pathogen classes, or components thereof. “Common stimulus-responsive” and “common regulated” genes can be used interchangeably.

Stimulus-specific genes are identified (i.e., selected, identified) by satisfying the following criteria: the ratio of gene expression score in response to the pathogens differs by greater than 2.5-fold compared to control media for all three donors or greater than 1.4-fold at two consecutive time points for all three donors. It is clear that the present invention can be used to generate databases comprising stimulus-responsive genes, stimulus-specific genes, and/or common stimulus-responsive genes (see Table 1, FIGS. 2 and 14). These databases will have many applications in medicine, research and industry. Specifically, the gene expression profiles described herein and identified by the methods of the present invention can be used to identify a pathogen or pathogen class to which a macrophage has been exposed.

Gene expression levels can be measured by a variety of methods known in the art. For example, gene transcription or translation products can be measured. Gene transcription products, i.e., RNA, can be measured, for example, by hybridization assays, run-off assays., Northern blots, or other methods known in the art.

Hybridization assays involve the use of oligonucleotide probes that hybridize to the single-stranded RNA transcription products. Thus, the oligonucleotide probes are complementary to the transcribed RNA expression product. Typically, s sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe such that sequence specific hybridization will occur. One of skill in the art will further know how to quantify the amount of sequence specific hybridization as a measure of the amount of gene expression for the gene was transcribed to produce the specific RNA.

A non-limiting example of a probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of a stimulus-specific gene expression product, or the probe can be the complementary sequence of such a sequence. Other suitable probes for use in the diagnostic assays of the invention are described herein.

The hybridization sample is maintained under conditions that are sufficient to allow specific hybridization of the nucleic acid probe to a specific gene expression product. “Specific hybridization”, as used herein, indicates near exact hybridization (e.g., with few if any mismatches). Specific hybridization can be performed under high stringency conditions or moderate stringency conditions. In one embodiment, the hybridization conditions for specific hybridization are high stringency. For example, certain high stringency conditions can be used to distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. et al., “Current Protocols in Molecular Biology”, John Wiley & Sons, (1998), the entire teachings of which are incorporated by reference herein). The exact conditions that determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70% o, at least about 80%, at least about 90% or at least about 95% or more identical to each other remain hybridized to one another. By varying hybridization conditions from a level of stringency at which no hybridization occurs to a level at which hybridization is first observed, conditions that will allow a given sequence to hybridize (e.g., selectively) with the most complementary sequences in the sample can be determined.

Exemplary conditions that describe the determination of wash conditions for moderate or low stringency conditions are described in Kraus, M. and Aaronson, S., 1991. Methods Enzymol., 200:546-556; and in, Ausubel, F. et al., Current Protocols in Molecular Biology, John Wiley & Sons, (1998). Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum mismatch percentage among the sequences that hybridize. Generally, doubling the concentration of SSC results in an increase in Tₘ of about 17° C. Using these guidelines, the wash temperature can be determined empirically for high, moderate or low stringency, depending on the level of mismatch sought. For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 minutes at 42° C.; and a high stringency wash can comprise washing in pre-warmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of complementarity between the target nucleic acid molecule and the primer or probe used (e.g., the sequence to be hybridized).

Specific hybridization, if present, is then detected using standard methods.

In another hybridization method, Northern analysis (see Current Protocols in Molecular Biology, Ausubel, F. et al., eds., John Wiley & Sons, supra) is used to identify the presence of a polymorphism associated with a susceptibility to osteoporosis. For Northern analysis, a test sample of RNA is obtained from the individual by appropriate means. Specific hybridization of a nucleic acid probe, as described above, to RNA from the individual is indicative of a particular allele complementary to the probe.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimetic having a peptide-like, inorganic backbone, such as N-(2-aminoethyl)glycine units, with an organic base (A, G, C, T or U) attached to the glycine nitrogen via a methylene carbonyl linker (Nielsen, P. et al., 1994. Bioconjug. Chem., 5:3-7). The PNA probe can be designed to specifically hybridize to a molecule in a macrophage RNA sample. Quantification of the PNA probe is therefore a measure of the level of expression of the target molecule for the probe.

In one embodiment, RNA from macrophages can be used to prepare “hybridization targets” according to published methods (Golub, T. et al., 1999. Science. 286:531-537). Expression profiles for multiple markers (e.g., stimulus-specific genes), or “target” RNA molecules, can be obtained by detecting the cellular level of RNA corresponding to each marker. This can be performed by quantitatively detecting specific RNA molecules by hybridization to complementary oligonucleotides. For example, hybridization assays to microarrays containing oligonucleotides complementary to specific marker mRNA transcripts arranged on gene chips available from Affymetrix, can be used to quantitatively detect RNA levels corresponding to thousands of markers in a single assay. Expression data can be obtained by assaying for the level of a gene expression product (e.g., RNA, peptide or protein). For example, a large expression database containing the expression profiles more than 16,000 markers from 218 tumor samples representing 14 common human cancer classes was created as a suitable database for use in methods described herein. Typical microarrays are attached to chips such as, for example, Affymetrix Hu6800 and Hu35KsubA GeneChips™. For these chips, arrays are scanned using commercially available Affymetrix protocols and scanners. Subsequent analysis can, for example, consider each probe set as a separate gene. Expression values for each gene are calculated using Affymetrix GeneChip™ analysis software. Such analysis can optionally include quality control for the quality and/or quantity of the RNA as determined by, for example, optical density measurements and agarose gel electrophoresis. Threshold limits can be set according to the practitioner, but a typically, scans are rejected if mean chip intensity exceeded 2 standard deviations from the average mean intensity for the entire scan set, if the proportion of “Present” calls was less than 10%, or if microarray artifacts were visible.

Alternatively, gene expression can be measured by assessing the level of a polypeptide or protein or derivative thereof translated from mRNA. Polypeptide gene expression products can be detected in protein binding assays, for example, antibody assays, or in nucleic acid binding assays, known in the art. For example, antibodies that specifically interact with the protein or polypeptide expression product of one or more stimulus-specific genes can be obtained using methods that are routine in the art. The specific binding of such antibodies to protein or polypeptide gene expression products can be detected and measured by methods known in the art.

In an alternate embodiment, the present invention is directed toward a method of identifying a pathogen comprising the steps of contacting one or more macrophages with a pathogen or with one or more immunogenic components thereof; isolating and labeling mRNA from the macrophages; detecting labeled mRNA from the macrophages such that a gene expression profile is produced; and analyzing the gene expression profile relative to one or more reference gene expression profiles such that at least one stimulus-specific gene is identified, thereby identifying a pathogen for which the stimulus-specific gene is specific. The present invention is useful in many clinical and non-clinical methods including but not limited to testing environmental, industrial and residential isolates for pathogens.

Diagnosis of infection is the first critical step in treatment of patients. In another embodiment, the present invention is directed toward a method of diagnosing infection in a mammal comprising the steps of isolating mRNA from a whole blood sample of a mammal; contacting the mRNA with at least one stimulus-responsive gene probe wherein hybridization of a stimulus-responsive gene probe to the mRNA is indicative of infection in the mammal. The ratio of gene expression of the stimulus-specific gene in comparison to a control, as described herein, identifies the pathogen (if present) responsible for the infection. Additionally, there are variations of the present invention that are well within the abilities of one of ordinary skill in the art. For example, to identify infection in a vertebrate, the gene probe can be a stimulus-specific or a common stimulus-responsive gene probe. The criteria for use of each probe type are described herein. Methods of determining hybridization of a probe to its target are well characterized in the art, for example, affinity chromatography, nucleic acid blotting, and the use of microarrays. The amount or level of hybridization of the stimulus-responsive probe can be positive or negative with respect to a reference, or a combination of both when measured in a time course. The expression or absence of expression of a gene can be indicative of infection.

Although the stimulus-responsive and stimulus-specific genes identified herein were identified in macrophages derived from monocyte precursors, clinical samples are more practically whole blood samples. As isolation of macrophages and monocytes would require more time than would be useful for an expedient diagnosis, the invention is particularly directed to diagnostic methods utilizing whole blood samples from a mammalian patient.

In another embodiment, the present invention is directed toward a method of diagnosing infection in a mammal comprising the steps of isolating proteins from one or more macrophages from said mammal; contacting said proteins with at least one stimulus-specific antibody wherein binding of a stimulus-specific antibody to said proteins are indicative of infection in said mammal. Methods of preparing protein extracts from cells and preparing antibodies are well known in the art. Antibodies can be polyclonal and/or monoclonal. Mixtures of stimulus-specific antibodies can also be used to detect stimulus-specific proteins. Lack of binding of a stimulus-specific antibody to the extracted proteins can be diagnostic of infection by a pathogen(s) or type of pathogens(s).

Particularly in cases where an infectious pathogen has not yet entered the blood stream, as is often the case with pneumonia, detection of protein products in the blood stream that result from the altered expression of stimulus-responsive or stimulus-specific genes is encompassed by the methods of the present invention.

In another embodiment, the present invention is directed toward a method of diagnosing infection by a specific pathogen in a mammal comprising the steps of isolating mRNA from one or more monocytes or macrophages in a mammal; determining a gene expression profile of at least one stimulus-specific gene wherein expression of a stimulus-specific gene is indicative of infection by a pathogen to which the stimulus-specific gene is specific. The profile of the stimulus-specific gene can be positive or negative with respect to a reference state, e.g., an uninfected state. “Positive” as defined herein means an increase in gene expression profile relative to a control gene expression profile. “Negative” as defined herein means a decrease in the gene expression profile relative to a control profile.

In another embodiment, the present invention is directed to a method of assessing prognosis for an infected individual by analyzing gene expression profiles of stimulus-responsive genes, wherein a specific expression profile is correlated with a clinical outcome. For example, the prognosis of a patient whose gene expression profile as determined by the present invention has a poor prognosis if their gene expression profile correlates with a pathogen-responsive gene expression profile. Alternatively, the prognosis of a patient whose gene expression profile as determined by the present invention correlates with a different reference gene expression profile of the present invention is indicative of a good prognosis. Such diagnostic methods allow for a more aggressive treatment for the patient.

Correlation is performed for a population of individuals who have been tested for gene expression profiles of stimulus-specific or common stimulus-responsive genes as described herein. To perform such analysis, the gene expression profile for at least one stimulus-specific or common stimulus-responsive gene is determined for a set of the individuals, some of whom exhibit a particular trait, and some of whom exhibit lack of the trait (e.g., pathogenesis). Correlation can be performed by standard statistical methods such as a Chi-squared test and statistically significant correlations between gene expression profile(s) and phenotypic characteristics are noted. For example, it could be found that the presence of Candida-specific gene expression profile correlates with eczema.

Such correlations can be exploited in several ways. In the case of a strong correlation between a gene-profile and a disease for which treatment is available, detection of a stimulus-responsive gene expression profile in a human or animal patient can justify immediate administration of treatment, the utilization of pathogen-specific treatment, or at least the institution of regular monitoring of the patient. Alternatively, the patient can be motivated to begin simple life-style changes (e.g., diet, exercise) that can be accomplished at little cost to the patient but confer potential benefits in reducing the risk of conditions to which the patient may have increased susceptibility by virtue of a stimulus-responsive gene expression profile. Identification of a stimulus-responsive gene expression profile in a patient correlated with enhanced receptiveness to one of several treatment regimes for a disease indicates that this treatment regime should be followed.

Alternatively, the present invention could be used to formulate a therapeutic regimen. As used herein “therapeutic regimen” is defined as treatment of a patient with pharmacological agents. In one embodiment, the present invention is directed to a method of formulating a therapeutic regimen comprising the steps of identifying the pathogen, and formulating a therapeutic regimen accordingly. Additionally, repeated assessment of a patient for a pathogen can determine the effectiveness of a therapeutic regimen which can then be altered accordingly. Methods of determining the effectiveness of a therapeutic regimen is known in the art.

Vaccine development is one of the most important prospective health safeguards in medicine. The present invention is suited for vaccine optimization. As used herein, “optimized” vaccines are more effective at preventing a pathogenic infection than non-optimized vaccines. In one embodiment, the present invention is directed to a method of vaccine optimization comprising the steps of contacting monocytes or macrophages with a test vaccine, and determining if an immune response is induced. The degree and specificity of the immune response is determined by comparison of the macrophage gene expression profile after exposure to the test vaccine to a reference gene expression profile of pathogen-responsive genes. Construction of vaccines and generation of variant test vaccines are known to those of skill in the art. As used herein “variant test vaccine” is a different composition of the original test vaccine. The optimized immune response refers to the immune response which promotes the highest level of pathogen immunity or killing. Methods of measuring immunity and killing of pathogens are well characterized in the art. The optimized vaccine is determined as described herein and defined as the test vaccine that elicits the strongest immune response. For example, the optimized vaccine should at least have the response produced by the standard vaccine for the pathogen, if available. The gene expression profile of the optimized vaccine can differ from other non-related vaccines.

Since macrophages mediate the innate and adaptive immune response, they are well suited to triggering an immune response to a specific antigen by the present invention. In one embodiment, the present invention is directed to an ex vivo therapeutic treatment for a pathogen comprising the steps of contacting a patient's macrophages with a pathogen or immunogenic components thereof such that said macrophages become activated; and returning activated macrophages to the patient such that the activated macrophages trigger an immune response against said pathogen.

In another embodiment, the present invention is directed to an ex vivo therapeutic treatment of tumors comprising the steps of contacting a patient's macrophages with tumor cells or immunogenic components thereof ex vivo such that said macrophages become “activated”; and returning activated macrophages to the patient such that the activated macrophages trigger an immune response against said tumor cells or components thereof. As used herein, “activated” refers to a state of macrophages such that the activated macrophage is capable of triggering an immune response. Such activation can involve, for example, antigen presentation, cytokine release, cytokine signaling, chemokine signaling, or activation of proteases. Any type of tumor cells may be used.

Macrophages are also involved in antigen tolerance, which is important in autoimmune diseases (such as, for example, arthritis) and in organ transplantation. In another embodiment, the present invention is directed to an ex vivo therapeutic treatment of autoimmunity comprising the steps of contacting a patient's macrophages with self-antigens or immunogenic components thereof ex vivo such that said macrophages become activated; and returning activated macrophages to the patient such that the activated macrophages do not trigger an immune response against said self-antigens or components thereof.

The most common problem associated with organ transplantation is graft-rejection. Graft-rejection is the process by which the transplanted tissue is recognized as non-self and triggers an immune response which leads to the destruction of the transplanted tissue. In another embodiment, the present invention is directed to an ex vivo therapeutic treatment of graft-rejection comprising the steps of contacting a patient's macrophages with graft-tissue or components thereof such that said macrophages become activated; and returning activated macrophages to the patient such that activated macrophages do not trigger an immune response against said graft-tissue or components thereof.

In another embodiment, the present invention is directed to a method of measuring the immune response to a stimulus comprising the steps of contacting macrophages with a stimulus, e.g., a pathogen; isolating mRNA from said macrophages and determining a gene expression profile such that at least one stimulus-responsive gene is identified which is indicative of an immune response. The gene expression profile of one or more genes can be measured as described herein and by methods known to one of skill in the art and include, but are not limited to, affinity chromotography, nucleic acid blotting, hybridization assays, PCR and the use of microarrays.

In another embodiment, the present invention is directed to a method of measuring the immune response to a stimulus comprising the steps of contacting macrophages with a stimulus; isolating and labeling mRNA from said macrophages; contacting a DNA microarray with labeled mRNA from the macrophages; and measuring the gene expression profile relative to control stimulus such that at least one stimulus-responsive gene is identified. In another embodiment, the present invention is directed to measuring the gene expression profile of macrophages in response to a stimulus comprising the steps of contacting macrophages with a stimulus; isolating and labeling RNA from the macrophages; contacting a DNA microarray with labeled mRNA from the macrophages; measuring the gene expression profile; and comparing the gene expression profile to a gene expression profile obtained from macrophages exposed to a control stimulus such that at least one stimulus-responsive gene is identified. The preferred source of macrophages is from monocytes derived from whole blood, although they can be isolated from any source including other body fluids, tissues, organs or obtained from a commercially available source. The methods of macrophage isolation and culture are well known to those of skill in the art and described herein. As used herein “immature” macrophages are defined as macrophages that have not been exposed to an antigen. Methods for production of immature macrophages are described herein.

Stimuli (e.g., pathogens) suitable for use in the present invention are bacteria, yeast, fungi, and viruses. Specific examples include, but are not limited to, gram-negative bacteria (Escherichia coli, enterohemorrhagic E. coli O157:H7 (EHEC), Salmonella typhi, and Salmonella typhimurium), gram-positive bacteria (Staphylococcus aureus and Listeria monocytogeness), mycobacteria (M. tuberculosis and Mycobacterium bovis bacille Calmette-Guérin (BCG)) influenza virus Candida albicans, lipopolysaccharide (LPS), polyI:C, yeast mannan and ds RNA (double-stranded RNA) or components thereof. Bacteria can be gram-positive or gram-negative. Viruses can be RNA (single-stranded or double-stranded) or DNA viruses. Bacteria, yeast, and viruses should be used at a concentration sufficient to ensure that the majority of macrophages are contacted with a microbe such that every cell responds (but still low enough that the microbe does not overgrow in the tissue culture plate and/or kill the macrophages). Bacteria and fungi can be used at a multiplicity of infection (MOI) from about 1 to 10,000. In particular embodiments, the bacterial or fungi can be used at MOI from about 1.5 to about 1000; about 2 to about 50; about 3 to about 20; or about 5 to about 10 MOI. Additionally, the stimulus can be physical, chemical, or electrical. Furthermore, the stimulus can comprise inorganic chemicals, organic chemicals or a combination thereof. The stimulus preferably elicits an immune response when exposed to macrophages. Methods of measuring an immune response are well characterized in the art.

In one embodiment of the present invention, macrophages are cultured with a stimulus. Subsequently, mRNA is isolated from macrophages at various time points after exposure. mRNA isolation from cells in culture is art standard (see Sambrook et al., Molecular Cloning: A Laboratory Manual, Cold Spring Harbor Laboratory Press; Ausubel, F. M., et al., Current Protocols in Molecular Biology, Greene Publishing Assoc. and Wiley-Interscience 1987, & Supp. 49, 2000, the teachings of which are incorporated herein by reference). mRNA is then labeled by standard methods (see above references). mRNA can be labeled with any appropriate molecule, for example fluorophores, biotin, radioactive nucleotide, and dye. In a preferred embodiment, mRNA is fluorescently labeled.

In one embodiment, labeled mRNA can be hybridized to DNA microarrays. The DNA microarray can be any high-density oligonucleotide microarray, for example, GeneChip™ HU 6800 (Affymetrix, Santa Clara, Calif.). Hybridization of labeled mRNA to the DNA microarray is known to those of skill in the alt (see Tamayo et al., 1999. Proc. Natl. Acad. Sci., USA, 96:2907-2912; Eisen et al., 1999. Methods Enzymol., 303:179-205).

Quantitation of gene expression profiles from the hybridization of labeled mRNA/DNA microarray is performed by scanning the microarrays to measure the amount of hybridization at each position on the microarray with an Affymetrix scanner (Affymetrix, Santa Clara, Calif.). For each stimulus, a time series of mRNA levels (C={C1,C2,C3, . . . Cn}) and a corresponding time series of mRNA levels (M={M1,M2,M3, . . . Mn}) in control medium in the same experiment as the stimulus is obtained. Quantitative data is then analyzed. Ci and Mi are defined as relative steady-state mRNA levels, where i refers to the ith timepoint and n to the total number of timepoints of the entire timecourse. μM and σM are defined as the mean and standard deviation of the control time course, respectively.

Alternatively, labeled RNA can be hybridized to a filter or other solid support containing target nucleic acids that comprise the pathogen-specific or common regulated genes described herein. Hybridization and wash conditions should be stringent enough to ensure specific binding between labeled RNA and target genes. Stringent hybridization and wash conditions are known to one of ordinary skill (see Sambrook et al., Molecular Cloning: A Laboratory Manual, Cold Spring Harbor Laboratory Press; Ausubel, F. M., et al., Current Protocols in Molecular Biology, Greene Publishing Assoc. and Wiley-Interscience 1987, & Supp. 49, 2000). Quantitation of specific hybridization can be performed by any suitable method including scintillation counting and densitometry.

The generation of the stimulus-specific databases by the present invention will provide a method for identifying a pathogen in a patient. In one embodiment, the present invention is directed to a method of identifying a pathogen comprising the steps of obtaining a whole blood sample (or isolated monocytes or macrophages), isolating and labeling mRNA derived from the sample cells, contacting a DNA microarray with labeled mRNA, and measuring and analyzing the gene expression profile such that at least one stimulus-specific gene is identified. The stimulus-specific gene is then used to search the stimulus-specific database such that a pathogen is identified. The stimulus-specific gene can be specific for more than one pathogen. Also, the pathogen can belong to a family of pathogens.

The invention is also directed to methods for screening for agents that elicit a pathogen-specific or pathogen immune response. The invention describes gene expression profiles for specific pathogens and identifying genes that are pathogen-specific or pathogen-responsive. A test agent can be put into contact with a macrophage under suitable test conditions, the macrophage mRNA can then be isolated, and a gene expression profile can be generated. A comparison of this gene expression profile to a pathogen-specific or pathogen-responsive gene expression profile will then allow one of skill in the art to determine whether the test agent is effective at inducing the desired immune response. For example, if the test agent induces a gene expression profile similar to that of the Gram-negative bacterially-induced reference gene expression profile, then it can be determined that the test agent is effective at inducing a Gram-negative-specific immune response.

The invention further provides kits comprising at least one stimulus-responsive or stimulus-specific indicator as described herein. Often, the kits contain one or more pairs of stimulus specific oligonucleotide probes. In some kits, the allele-specific oligonucleotides are provided immobilized to a substrate. For example, the same substrate can comprise stimulus specific oligonucleotide probes for detecting at least 1, 5, 10 or all of the stimulus specific gene expression products described herein. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, reagents of the kit are packaged together with instructions for carrying out the methods.

The invention further relates to an oligonucleotide microarray having immobilized thereon a plurality of oligonucleotide probes that serve as stimulus-specific and/or stimulus-responsive probes. For example, the microarray can contain one or more stimulus specific probes for a gene listed in Table 1. The preparation of such oligonucleotide microarrays is well known in the art.

The present invention encompasses kits for all of the disclosed embodiments. The present invention provides a list of genes regulated in response to gram-negative bacteria (Escherichia coli, enterohemorrhagic E. coli O157:H7 (EHEC), Salmonella typhi, and Salmonella typhimurium), gram-positive bacteria (Staphylococcus aureus and Listeria monocytogenes), mycobacteria (M. tuberculosis and Mycobacterium bovis bacille Calmette-Guérin (BCG)), and components thereof. The methods of the present invention can be used to identify pathogens and diagnose infections. Additionally, the present invention provides methods for optimizing vaccines and treating autoimmune diseases such as arthritis and graft-rejection.

While this invention has been particularly shown and described with references to preferred embodiments thereof, it will be understood by those skilled in the art that various changes in form and details may be made therein without departing from the scope of the invention encompassed by the appended claims. The teachings of all references cited herein are incorporated herein by reference.

### EXEMPLIFICATION

### Example 1

To improve the understanding of host defenses and pathogenesis, the effect of different bacteria on gene expression in human macrophages was investigated. A detailed, comparative evaluation was made of the transcriptional responses of macrophages to a variety of bacteria, including a number of pathogens (Schwandner, R. et al., 1999. J. Biol. Chem., 274:17406-17409). An in vitro model system of human macrophages was used in which the macrophages were matured from commercially available monocytes. These monocytes are elutriated from leukopheresis packs of healthy donors (ABI Technologies, Columbia, Md.). Macrophage gene expression after exposure to bacteria was measured over a 24 hour time course using high density DNA microarrays (Affymetrix, Santa Clara, Calif.). These experiments characterized the innate immune response to a variety of bacteria, defining shared and distinct macrophage responses.

Analysis of the macrophage gene expression data revealed robust gene expression changes that were induced by all the bacteria tested. This signature was also induced by bacterial components known to be agonists for Toll-like receptors (TLR), suggesting the signature represents the transcriptional end result of the TLR/NFκB pathway. Identifying this common response helped to highlight the differences induced by distinct bacteria.

Organism-specific signatures were identified that provided clues to understanding pathogenesis. Mycobacterium tuberculosis poorly induced two cytokines, IL-12 and IL-15, compared to the other bacteria. These novel findings from the microarray data led to the discovery that M. tuberculosis actually inhibits production of IL-12, suggesting how the tubercle bacillus might undermine host defenses.

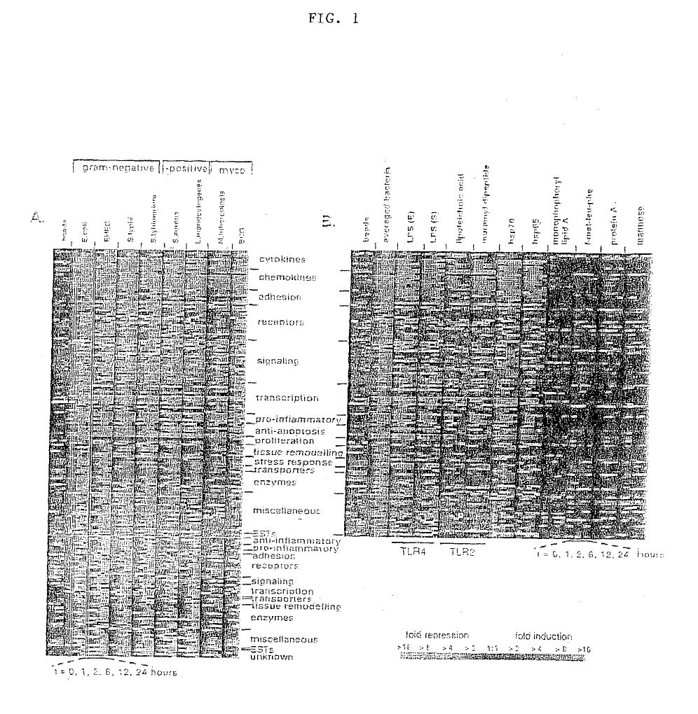

Another analysis has disclosed macrophage responses that distinguish between exposure to Gram-negative and Gram-positive organisms. This analysis was conducted as follows. The average change in gene expression of macrophages exposed to bacteria, compared to untreated macrophages, was calculated at four time points for all time courses using Gram-negative organisms (n=9). A similar calculation was done for time courses using Gram-positive organisms (n=4). Genes that were significantly different between the two averages were identified by t test (p<0.005). This analysis revealed 42 genes differentially expressed in macrophages exposed to Gram-negative and Gram-positive bacteria (FIG. 1, Table 1). The majority of the signature is composed of genes expressed more highly in macrophages exposed to Gram-negative bacteria (FIG. 1). Three genes of this cluster were particularly interesting because they are chemokines: I-TAC, MIG, CXCL10. An ELISA was developed to measure protein levels of 1-TAC in macrophage supernatants. Gram-negative organisms induced 1-TAC production whereas Gram-positive organisms did not. Similar responses were observed when dendritic cells were treated with LPS from Gram-negative bacteria but not lipoteichoic acid from Gram-positive bacteria.

Inspection of the microarray data also suggested genus-specific responses. IFN-β was induced by Escherichia but not by Salmonellae. This finding was confirmed by ELISA of supernatants. In sum, the microarray data collected to date provide multiple possibilities for diagnostic biomarkers.

The TLR4 core signature is the most likely transcriptional outcome of this Mal/TIRAP pathway.

Research and Methods

The experiments described herein are designed to evaluate biomarkers for diagnosis. The studies build on the body of gene expression data generated with DNA microarrays and seek to derive direct clinical benefit from these data. The plan is first to characterize and validate a panel of biomarkers in vitro and second to test their expression during infection in vivo.

The experiments are designed to evaluate a panel of biomarkers for Gram-negative infection. Although the presence of I-TAC protein appears to identify cells exposed to Gram-negative bacteria, it is possible that this protein may not be detectable in all patients. It would be desirable to have a series of biomarkers that distinguish between infections by these two classes of bacteria, thereby increasing confidence in a diagnosis. Moreover, the gene expression data associate IFN-β production with E. coli.

Identification of genus-specific markers would be an important step to developing single biomarkers or patterns of biomarkers that are diagnostic of specific organisms. To increase the panel of biomarkers of Gram-negative infection, two other candidate genes are evaluated. CXCL10 and MIG genes were strongly expressed in macrophages exposed to Gram-negative bacteria. ELISAs are developed for CXCL10 and MIG protein. Monoclonal capture antibodies, biotinylated polyclonal capture antibodies, and positive control recombinant proteins are purchased from R&D Systems (Minneapolis, Minn.). CXCL10 and MIG levels are measured to determine if they discriminate between Gram-positive and Gram-negative infections. Supernatants derived from multiple donors' macrophages exposed to a variety of bacteria are on hand. These resources permit rapid assessment of the candidate biomarkers by comparison to reference expression profiles.

Analysis of the gene expression profiles of macrophages exposed to Gram-negative bacteria suggests that IFN-β levels discriminate between exposure to Escherichia and Salmonella. Stocks of other Gram-negative organisms are obtained to evaluate how closely IFN-β is associated with Escherichia. For example, a variety of organisms such as Pseudomonas aeruginosa, Klebsiella pneumoniae, and Stenotrophomonas maltophilia are obtainable from strain collections (American Type Culture Collection) or other researchers at the hospital. Virulent type strains and sequenced strains are used when possible. Frozen stocks of bacteria are tested for viable colony-forming units. Established protocols for in vitro infection (Schwandner, R. et al., 1999. J. Biol. Chem., 274:17406-17409) are used, adding approximately ten bacteria per macrophage. Supernatants are sterilized by passing through a 0.2 μm filter and are tested for cytokine production. ELISA kits for IFN-β are commercially available.

It is important to identify biomarkers that affirm the diagnosis of infection by Gram-positive bacteria. It is possible that the Student's t-test analysis is not able to detect expression changes attributable to Gram-positive organisms because fewer experiments are available for this analysis (n=4). To identify Gram-positive-specific biomarkers, alternative analyses are conducted. One approach is to lower the significance level in the t-test. This approach, however, may result in an unacceptable number of false positive candidates. Because the biomarkers identified thus far have been cytokines, a simple alternative analysis is to survey cytokines represented on the array for differences that may have failed to meet the threshold for significance. A more sophisticated analysis, such as significance analysis of microarrays (Yoshimura, A. et al., 1999. J. Immunol., 163:1-5) and Pearson correlation tests, may also yield clues to biomarkers specific for Gram-positive infections. While the protein assays are being developed for the biomarkers discussed above, reevaluation of the expression data for additional biomarkers continues.

Characterization of the biomarkers in vitro is reinforced by evaluating their predictive value during infections in vivo. The first step is to evaluate patients with bacteremia to increase the likelihood of detecting biomarkers at the site of the infection, i.e., the bloodstream. Ultimately, patients with distant, non-bacteremic infections must be tested to determine if serum biomarkers are diagnostic of localized infections.

Initially, a test for biomarkers in patients with known infections can be conducted. Approval has been obtained from Massachusetts General Hospital's Institutional Review Board to harvest plasma and serum specimens from patients with positive blood cultures. Samples are taken from discarded chemistry specimens that are drawn around the time the blood cultures were obtained. Candidate Gram-negative biomarkers, I-TAC, CXCL10, and MIG, are measured to determine those that are expressed most robustly in vivo. It is also of interest to determine whether IFN-β can be detected in vivo. Specimens are tested for the presence of IFN-β. Based on previous work (Poltorak, A. et al., 1998. Science, 282:2085-2088), an initial assessment of biomarker expression is conducted with samples from one to two dozen patients with Gram-negative and Gram-positive bacteremias.

One potential pitfall is that the concentration of biomarkers circulating in the bloodstream is below the limits of detection in a standard ELISA. Serum levels of cytokines can be one to two orders of magnitude lower than levels in vitro. The sensitivity an ELISA can be greatly enhanced using chemiluminescent substrates, such as Supersignal Femto substrate (Pierce Chemical, Rockford, Ill.). With limits of detection as low as femtograms/mL, there is improved sensitivity to detect circulating biomarkers. Also, the levels of biomarkers are determined by the rates of production and removal. It is helpful to test multiple plasma specimens drawn from the same patient at various times to identify peak levels in vivo.

Another intriguing application biomarkers is to diagnose the etiologic agent at a distant site of infection. It is not known if extravascular infections lead to biomarker accumulation within the bloodstream. One approach is to focus on blood cultures that fail to grow an organism, which may be reported as “no organism seen” (NOS), and when a bacteriologic diagnosis is made elsewhere, for example in sputum or urine. A preliminary investigation of those blood cultures reported as NOS will be conducted. If there is a positive culture from an alternative site, such as sputum, serum samples will be tested for biomarkers.

The experiments described above evaluate biomarkers to diagnose particular infections. These studies are the first step to developing a new approach to early diagnosis of infections. This approach may be particularly useful when the etiologic agent cannot be identified, such as culture-negative endocarditis. If successful, the results of these experiments would support further investigation of host-pathogen interactions in vitro to generate more candidate biomarkers and test their clinical utility. A panel of biomarkers capable of identifying the class, genus, or species of infectious agent should aid clinicians managing patients with infectious diseases.

### Example 2

Materials and Methods

Macrophage culture. Freshly elutriated human monocytes (>95% pure) were purchased from ABI (Columbia, Md.). Monocytes were cultured at a density of 2×10⁷ cells/10 mL of DMEM (InVitrogen, Carlsbad, Calif.) with 20% FCS (Intergen, Purchase, N.Y.), 10% human serum (Nabi, Boca Raton, Fla.) and 50 μg/mL gentamicin (Invitrogen) in Primaria T-25 flasks (Becton Dickinson, Franklin Lakes, N.J.) for 5 days at 37° C., 5% CO₂, on days 5 and 7, half of the media was removed and replaced with media lacking FCS. Media on the cultured macrophages was changed to 5 mL of DMEM with 1% human serum on day 9, one hour before experiments were begun.

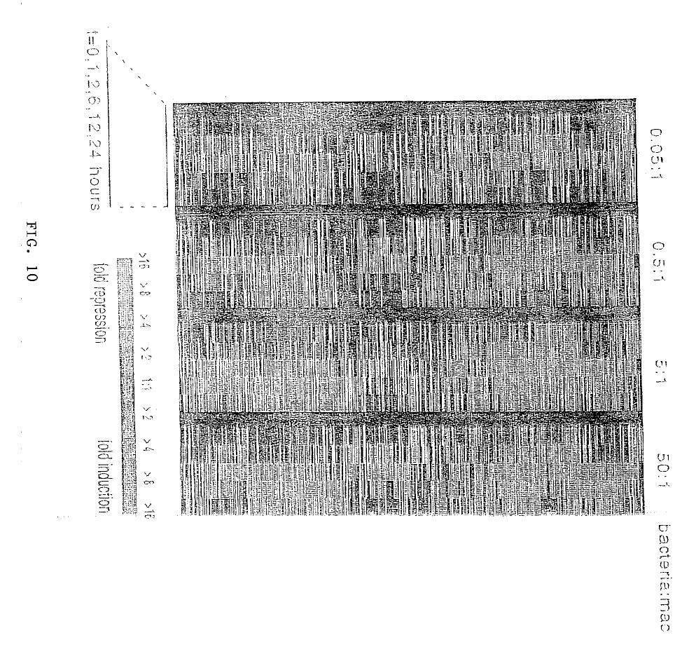

Macrophage exposure to bacteria. Latex beads (0.8 μm, Sigma, St. Louis, Mo.) were washed with ethanol and added. Bacteria were diluted in media to 50 μl containing 10⁸-10⁹ bacteria, to give multiplicities of infection (MOIs) of 5:1 to 50:1, and added to cultures of macrophages. Dilutions were plated on agar plates and colonies were counted to confirm the accuracy of dilution and viability of bacteria. This range of MOIs was selected because preliminary experiments revealed that, although the expression profiles elicited by different MOIs are largely similar, some genes with low levels of expression are not robustly detected at low MOIs (FIG. 10). Media was removed at 4 hours and cells were washed with Hank's Balanced Salt Solution (HBSS, Invitrogen) and fed with 5 mL of DMEM with 1% human serum and 50 μg/mL gentamicin. Staphylococcus aureus strain ISP794, derived from strain 8325, and Listeria monocytogenes strain EGD were grown overnight in brain-heart infusion broth, pelleted, and resuspended in 0.5× volume of 20% glycerol to make frozen stocks that were thawed for the experiments. Mycobacterium tuberculosis Erdman stain, obtained from TB Research Materials and Vaccine Testing Contract, and Mycobacterium bovis BCG (ATCC 35734) were grown in Middlebrook 7H9 broth with 0.5% glycerol, 0.05% Tween 80, and ADC enrichment (Becton Dickinson, Franklin Lakes, N.J.). A 6-7 day old culture was used for infection. Escherichia coli strain sd-4 (ATCC #11143) was grown in Luria-Bertani medium with streptomycin and frozen stocks were created. The Salmonella typhi (Quailes strain), and S. typhimurium (ATCC #14028) and enterhemorrhagic E. coli O157:H7 (EHEC) were provided as frozen stocks and thawed on the day of the experiment.

Macrophage exposure to bacteria components. Macrophages were stimulated with isolated and purified bacterial components in media containing 10% human serum over the entire 24-hour time course. Endotoxin-free status was verified for all components, and quantitated for LPS and hsps, using the Associates of Cape Cod (Falmouth, Mass.) gel-clot assay at a sensitivity of 0.3 Eu. LPS from E. coli (Sigma, L2880) and Salmonella (Sigma, L4774) were added at a final concentration of 1 μg/mL. Lipoteichoic acid (Sigma, L-2515) and muramyl dipeptide (Sigma, A-9519) were added at 10 μg/mL and 100 μg/mL, respectively. Formyl-methionine-leucine-phenylalanine (Sigma, F-3506) was added to 100 nM, protein A to 10 μm/mL, and D-(+)-mannose (Sigma, M-8296) to 25 mM. Staphylococcus protein A (539202) was purchased from Calbiochem (San Diego, Calif.). Recombinant mycobacterial hsps were the kind of gift of StressGen (Victoria, British Columbia) and were used at 10 μg/mL.

cRNA target preparation and array hybridization. After incubation, the supernatant was recovered from the flasks and RNA was harvested from the macrophages using 2 mL TriReagent (Molecular Research Center, Inc., Cincinnati, Ohio) according to the manufacturer's protocols. Total RNA was processed and hybridized to Hu6800 GeneChips according to standard Affymetrix protocols.

Data analysis. The data were analyzed using a custom-built Oracle database, ChipDB. Fluorescence intensities were normalized to median array intensities for all conditions tested on cells from a single donor, floored at 50, and fold-change was calculated relative to duplicate time zero controls. Data were considered significant when 1) expression changed by at least two-fold at two consecutive time points, or ten-fold (activation program) or a 3-fold (differential gene expression) at a single time point, and 2) increased gene expression included at least one present call (Affymetrix algorithm), or both zero time points were present when gene expression decreased. Additional details are available on the web site supporting this manuscript at the internet site, web.wi.mit.edu/young/pathogens.

Bacteria-Induced Macrophage Activation Program

Human macrophages derived from primary monocytes were exposed to bacteria and bacterial components, and the resulting expression levels of 6800 genes were monitored over a 24-hour time course using high density DNA microarrays. The eight bacteria studied were drawn from three broad classes with different cellular components and pathogenesis: gram-negative bacteria (Escherichia coli, enterohemorrhagic E. coli O157:H7 (EHEC), Salmonella typhi, and Salmonella typhimurium), gram-positive bacteria (Staphylococcus aureus and Listeria monocytogenes), and mycobacteria (M. tuberculosis and Mycobacterium bovis bacille Calmette-Guérin (BCG)).

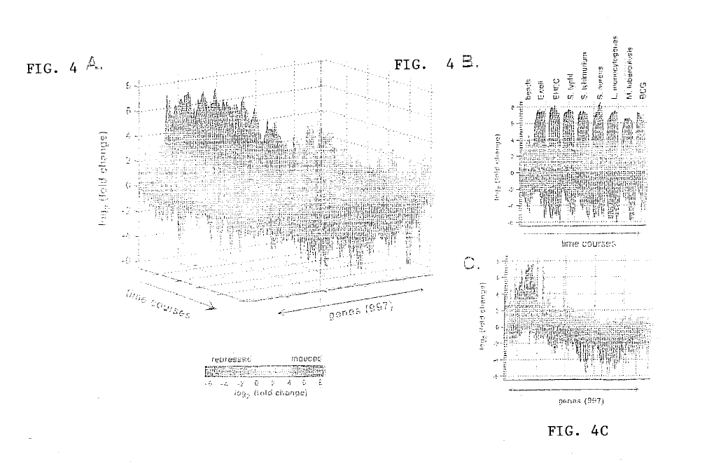

Analysis of macrophage gene expression data showed that the expression of 977 genes significantly changed upon exposure to one or more of the bacteria. Unsupervised hierarchical clustering of these genes revealed prominent groups of genes that had similar changes in expression. Despite the diversity of the bacteria studied, a shared transcriptional response was elicited, consisting of 132 genes induced and 59 repressed (FIG. 1A, the entire data set is displayed in FIGS. 4A-4C). Latex beads failed to induce comparable expression changes (FIG. 1B). The changes in gene expression induced after exposure to bacteria contained, as expected, pro-inflammatory genes, including many cytokines and chemokines (FIG. 1A, Table 1). The majority of this transcriptional response, however, is comprised of genes that are involved with the interaction between macrophages and their environment. A large number of receptors, signaling molecules, and transcription factors were differentially regulated by macrophages upon exposure to bacteria, as were adhesion molecules, genes involved in tissue remodeling, enzymes and anti-apoptotic molecules. For example, macrophages up-regulated many of the NF-κB pathway members as well as receptors for chemokines (CCR6 and CCR7) and interleukins (IL-7R, IL15RA), and down-regulated molecules necessary to respond to TGF-β (Table 1). This collection of genes was defined as the macrophage activation program since it represents signaling pathways and induced functions that are triggered upon exposure to gram-negative, gram-positive and mycobacteria.

TABLE I
Genes of the activation program.

| INDUCED | | | REPRESSED | |
|---|---|---|---|---|
| Cytokines | Signaling | Proliferation | Anti-Inflammatory | Miscellaneous |
| GCSF | CNK | BTG1 | GCHFR | PURA |
| GMCSF | DUSP1 | BTG3 | CAT | MYO1E |
| IL12B | DUSP2 | TNFRSF9 | LTA4H | VCL |
| IL1RN | DUSP5 | TNFSF9 | Pro-Inflammatory | IV12B |
| IL6 | EBI2 | Tissue Remodeling/clotting | CD14 | ADFP |
| IL6 | GBP1 | DAP | ALOX5 | MNDA |
| PBEP* | HCK | MMP1 | MCP1 | STAB1 |
| ProIL1B | INHBA | MMP10 | Adhesion | TGFBI |
| TNFA | JAG1 | MMP14 | PECAM1 | ESTs |
| Chemokines | KYNU | SERP1NB2 | SPARC | KIAA0022 |
| IL8 | LIMK2 | SERPINB8 | Receptors | Unknown |
| IL8 | MAP2K3 | Stress response | RARA | AD000092 |
| IP10 | MAP3K4 | GADD45A | TNFRSF1A | D87075 |
| MCP1 | MINOR | HSPA1A | ENG | U79288 |
| MGSA | NAF1 | SOD2 | L77730 | P311 |
| MIP1A | NFKB1 | SOD2 | CD36L1 | HG2090-HT2090 |
| MIPIB | PDE4B | Transporters | CD163 | HG2090-HT2090 |
| MIP2A | PPP3CC | ATP2B1 | TGFBR2 | HG2090-HT2090 |
| MIP2B | PTPN1 | NRAMP2 | GSF1R | |
| RANTES | TRAF1 | SLC7A5 | MRC1 | |
| Adhesion | TRIP10 | Enzymes | CD32 | |
| CD44 | Transcription | ADA | P2RX1 | |
| CD44 | DSCR1 | AMPD3 | Signaling | |
| ICAM1 | ELF4 | beta-1,4-galatosyl transferase | SRPK2 | |
| IFITM1 | ETS2 | BF | SLA | |
| LAMB3 | IRF1 | CKB | MERTK | |
| NINJ1 | IRLB | GJB2 | DAB2 | |
| TNFA1P6 | JUNB | GLCLR | Transcription | |
| Receptors | MRF-1 | HSD11B1 | SF3A3 | |
| ADORA2A | NFKBIA | INDO | EGR2 | |
| CCR6 | NFKBIE | MTF1 | NFATC3 | |
| CCR7 | NFKBp50 | Miscellaneous | MX11 | |
| CCRL2 | STAT4 | ADM | FOS | |
| DTR | STAT5A | ARHH | Hbrm | |
| EBI3 | TSC22 | B4-2 | Transporters | |
| HM74* | XBP1 | BRCA2 | SLC29A1 | |
| ILI5RA | ZFP36 | CD83 | Tissue remodeling/clotting | |
| IL7R | Pro-Inflammatory | GEM | UPA | |
| LDLR | COX2 | GOS | FGL2 | |
| P2RX7 | COX2 | GYPC | TBXAS1 | |
| P2XR | COX2 | H2AFO | Enzymes | |
| PLAUR | GCH1 | HIVEP2 | SGSH | |
| PVR | PTX3 | ISG15 | PPP2R5C | |
| SLAM | Anti-apoptotic | ISG20 | IDH2 | |
| TNFRSF5 | BIRC2 | MACMARKS | LPL | |
| TXN | BIRC3 | MIG2 | MPI | |
| | BIRC3 | MXJ | PYGL | |
| | CFLAR | RCN1 | HSD17B4 | |
| | IER3 | SDC4 | RNASE6 | |
| | TNFAIP3 | SNL | GALC | |
| | | TNFAIP2 | GLCLC | |
| | | TSSC3 | RNASE1 | |
| | | ESTs | ME1 | |
| | | K1AA0105 | | |
| | | KIAA0172 | | |

The genes of the activation program are listed in the order displayed in FIG. 1 and are categorized by functional class. Several genes are repeated because they appear more than once on the array.

Next, the question of whether specific bacterial components could elicit the activation program was addressed. Macrophages were cultured with purified components specific for gram-negative bacteria (lipopolysaccharide. (LPS)), gram-positive bacteria (lipotichoic acid (LTA) and protein A) and general to all 3 classes studied (muramyl dipeptide (MPD), heat shock proteins (HSP65 and HSP70), formyl-methionine-leucine-phenylalanine (f-MetLeuPhe), and mannosylated proteins (D-(+)-mannose)). LPS, LTA, MDP, hsp65 and hsp70 induced the majority of the activation program (FIG. 1B). The other components failed to induce this set of gene expression changes (FIG. 1B), as did monophosphoryl lipid A (detoxified lipid A). Thus, some, but not all, bacterial components are capable of inducing the activation program.

M. tuberculosis-Specific Macrophage Gene Expression Changes

In order to identify the alterations in the host macrophages expression profiles due to specific organisms, a difference index was devised. This analysis focused on M. tuberculosis, E. coli, and S. aureus because multiple, independent time course experiments were performed for each of these organisms (FIG. 2A). Using this index, several differentially regulated genes with positive and negative effects on host defenses were identified. M. tuberculosis poorly induced the expression of interleukin-12 (IL-12) p40 and IL-15, as described in greater detail below. STAF50, a transcriptional repressor of HIV LTR, and STAT2, a transcription factor critical to responsiveness to interferons, and IL-10RA were induced in macrophages by E. coli. Similarly, S. aureus induced a pro-inflammatory lysophospholipase, HU-K5, but also down-regulated several cytoskeletal components and up-regulated a negative regulator of G-protein signaling. Thus, organism-specific macrophage gene expression changes were discernable.

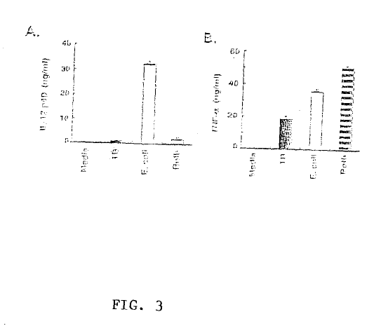

Because of the impact of tuberculosis on world health, we focused additional experiments on the gene expression changes induced by M. tuberculosis to test the difference index and evaluate clues to pathogenesis. It was striking that the expression levels of genes encoding two proteins critical for host defense and adaptive immunity, IL-12 and IL-15, were differentially regulated. The induction of these genes was significantly lower following exposure to M. tuberculosis than other bacteria (FIG. 2B). Although IL-12 met the criteria for inclusion in the macrophage activation program (Table 1), this subsequent analysis disclosed substantial differences in the levels of IL-12 expression by macrophages exposed to M. tuberculosis and other bacteria. As predicted by the microarray data, supernatants from macrophages stimulated by M. tuberculosis contained less IL-12 p40 than macrophages exposed to E. coli (FIG. 3A). Macrophages exposed to M. tuberculosis also secreted less IL-12 p70 and IL-15 protein than macrophages cultured with E. coli (FIG. 2B).

Since the lower levels of IL-12 in macrophages exposed to M. tuberculosis could be due to active repression of IL-12 production by M. tuberculosis, a co-culture experiment was performed. Macrophages were exposed to M. tuberculosis, to E. coli, or to both simultaneously. Macrophages exposed to a mixture of M. tuberculosis and E. coli produced low levels of IL-12, comparable to macrophages exposed to M tuberculosis alone and substantially less than macrophages exposed to E. coli alone (FIG. 3A). Although M. tuberculosis inhibited the production of IL-12 levels normally induced by E. coli, this inhibition did not extend to the production of TNF-α (FIG. 3B). These findings indicate that M. tuberculosis alters the macrophage activation program and dominates the stimulatory effects of E. coli exposure.

### Example 3

Cells and Reagents

Gene expression data of macrophage responses to bacteria and bacteria components are from experiments conducted previously (Nau, G. et al., 2002. Proc. Natl. Acad. Sci. USA, 99:1503-1508). To generate macrophages, 2×10⁷ human monocytes (Advanced Biotechnologies, Inc., Columbia, Md.) were cultured in 10 mL of DMEM (Invitrogen, Carlsbad, Calif.) with 20% FCS (Intergen, Purchase, N.Y.), 10% human serum (Nabi, Boca Raton, Fla.) and 50 μg/mL gentamicin (Invitrogen) in Primaria T-25 flasks (Becton Dickinson, Franklin Lakes, N.J.) for 5 days at 37° C., 5% CO₂. On days 5 and 7, one half of the media was removed and replaced with media lacking FCS. Media on the cultured macrophages was changed to 5 mL of DMEM with 1% human serum on day 9, one hour before beginning experiments with bacteria. Media was changed to 5 mL of 10% human serum 16 hours prior to initiating experiments using bacterial components and cytokines. Some follow up experiments were conducted in microtiter plates. For these experiments, monocytes were cultured 7 days in 60 mm tissue culture dishes (Falcon #3002, Becton-Dickinson, Lincoln Park, N.J.) and macrophages were removed using PBS containing 5 mM EDTA and 4 mg/mL lidocaine before seeding in Primaria microtiter plates (Falcon) at a density of 2.5×10⁴ to 1.0×10⁵ macrophages per well. For whole blood experiments, peripheral blood was harvested in the presence of EDTA, washed twice with PBS, and resuspended to twice the volume with DMEM containing 10% human serum (Nabi, Boca Raton, Fla.). Two mL of these mixtures were plated per well of a 6-well Primaria plate (Falcon). Supernatants were harvested after 24 hours of culture with 2×10⁷ bacteria. Phlebotomy protocols were approved by the MIT institutional review board.

Human cells were activated with a variety of stimuli. Bacteria were thawed from frozen stocks for inoculation. Escherichia coli strain sd-4 (ATCC #11143) was grown in Luria-Bertani medium with streptomycin and frozen stocks were created from stationary phase cultures as described (Nau, G. et al., 1997. Proc. Natl. Acad. Sci. USA, 94:6414-6419). Glycerol stocks from overnight cultures of Staphylococcus aureus strain ISP794, derived from strain 8325, Listeria monocytogenes strain EGD, Salmonella typhi (Quailes strain), S. typhimurium (ATCC #14028) and enterohemorrhagic E. coli O157:H7 (EHEC) were prepared from cultures grown in brain-heart infusion broth. A multiplicity of infection of 5-50:1 (bacteria:macrophage) was used. After 4 hours, flasks were washed with warm Hank's balanced salt solution to remove extracellular bacteria and 5 mL of fresh culture medium was added. Bacteria components were from Sigma (St. Louis, Mo.): LPS from E. coli (L-2880), final concentration of 1 μg/mL; lipoteichoic acid (L-2515) and muramyl dipeptide (A-9519) final concentration of 10 μg/mL and 100 μg/mL, respectively; formyl-methionine-leucine-phenylalanine (F-3506) final concentration 100 nM. Human IFN-α was from Schering-Plough (Kenilworth, N.J.), IFN-β from Bioscience International (Camarillo, Calif.), and IFN-γ from R&D Systems (Minneapolis, Minn.). For experiments using bacterial components or cytokines, 5 mL of fresh medium were placed on flasks the night prior to the experiment. Components or cytokines were added directly to the flasks; these flasks were not washed at 4 hours. Monoclonal anti-IFNAR2 was from Calbiochem (San Diego, Calif.) and isotype controls were from R&D Systems.

cRNA Target Preparation and Array Hybridization.

After incubation, supernatants were removed from the flasks and passed through a 0.2 μm filter. Macrophages were lysed with 2 mL of TriReagent (Molecular Research Center, Inc., Cincinnati, Ohio) and total RNA was isolated with chloroform and isopropanol using the manufacturer's protocol, with Eppendorf Phase Lock Gel Heavy tubes (Cat. #0032 005.152, Brinkman Instruments, Westbury, N.Y.) to aid isolation of the aqueous phase. First strand cDNA was synthesized from 20 μg of total RNA, T7-poly-dT primer (10 μM final, Sigma Genosys), and Superscript II (Invitrogen). Second strand cDNA was synthesized DNA ligase 10 units, DNA polymerase 1 (40 units), and RNAse H (2 units) (all from Invitrogen). After phenol-chloroform extraction and precipitation, the entire cDNA product was used for in vitro transcription with biotinylated-CTP and UTP (Einzo Diagnostics, Farmingdale, N.Y.) and a Megascript T7 kit (Ambion, Austin, Tex.). The resulting cRNA was purified using a Qiagen RNAeasy kit (Valencia, Calif.). Typically 60-80 μg of cRNA was obtained. The process was repeated for samples yielding less than 50 μg of cRNA.

For hybridization, 15 μg of cRNA were fragmented per Affymetrix protocols and 10 μg were incubated overnight to HuFL GeneChips™. These microarrays contain over 6800 unique human genes and ESTs. The total number of genes on the array (7070) includes multiple representations of some genes. After removal of cRNA, microarrays were washed and sequentially incubated with streptavidin coupled to phycoerythrin, biotinylated anti-phycoerythrin, and streptavidin-phycoerythrin according to Affymetrix protocols. Fluorescence intensities were measured with Affymetrix scanners. Data from samples that had fewer than 1500 “present” calls (a measure of scan quality derived from Affymetrix software) were discarded and cRNA was re-made or, in some cases, the experiment was repeated from the beginning. These protocols are available in detail on the world wide web site,

staffa.wi.mit.edu/cgi-bin/young_public/navframe.cgi?s=8&f=protocols.

Analysis of Gene Expression Data.

To compare the effects of Gram-negative bacteria and Gram-positive bacteria, the following analysis was conducted. Fluorescence intensities were normalized to median array intensities for all conditions tested on cells from a single donor, floored at 50, and fold change was calculated relative to duplicate time zero controls. The average change in gene expression (log₂) was then calculated at each time point for all the time courses using Gram-negative organisms (9 time courses). A similar calculation was done for time courses using Gram-positive organisms (4 time courses). Two or three time courses per bacterium were conducted on different donors. Genes were considered significantly different between the two average time courses if there was at least a 2-fold difference in expression levels and p<0.01 by Student's t-test in at least one of the 4 time points measured (1, 6, 12, 24 hours). To test these selection criteria, datasets were randomized within each time point to create new groups of data irrespective of the bacteria used to activate the macrophages. Application of the selection criteria to these shuffled datasets failed to identify significant differences in gene expression between the new groups of data.

ELISAs.

Supernatants were harvested after 24 hours of culture. For I-TAC, the capture antibody (MAB672) and detection antiserum (BAF672) were obtained from R&D Systems and were used at 2 μg/mL (capture) and 100 ng/mL (detection). The ELISA was conducted using the standard R&D Systems protocol for matched antibody pairs. IL-12 p40 was measured using the R&D Systems DuoSet. Levels of 1L-12 p70 was measured with the R&D Systems High Sensitivity (HS) Quantikine Kit or DuoSet as indicated in figure legends.

Results

Differential Gene Expression in Human Macrophages

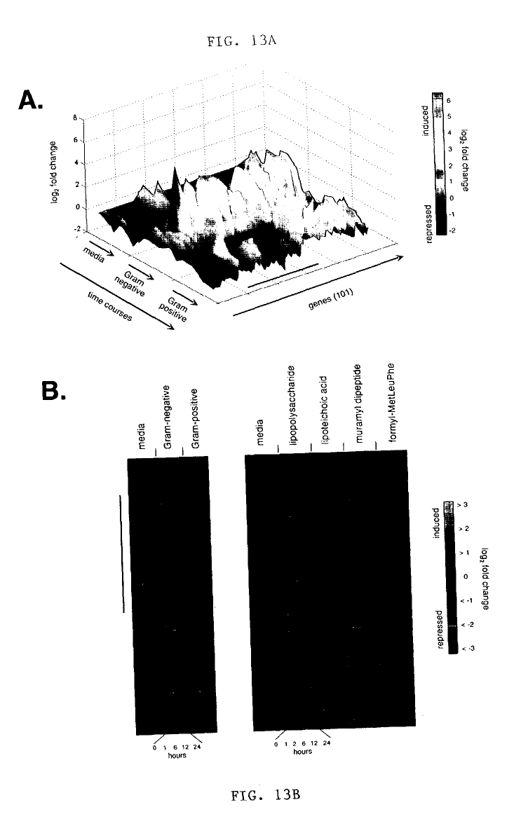

The response of human macrophages to a variety of whole bacteria was explored to determine if differential signals could be correlated with a particular host TLR. Monocyte-derived macrophages were exposed to bacteria over a 24 hour time course, and macrophage gene expression was measured using Affymetrix GeneChips. The same expression data were re-analyzed to identify macrophage gene expression changes in response to Gram-negative organisms (E. coli, enterohemorrhagic E. coli O157:H7, S. typhi, S. typhimurium) compared to those induced by Gram-positive organisms (S. aureus, L. monocytogenes). This analysis identified 101 genes that were significantly different in macrophages exposed to these two classes of bacteria. The time course data for these genes were subjected to hierarchical clustering (Eisen, M. et al., 1998. Proc. Natl. Acad. Sci. USA, 95:14863-14868) and are displayed in FIG. 13A. For comparison, the average gene expression changes of four time courses of untreated macrophages are shown. Most of the differentially regulated genes were more highly expressed in macrophages exposed to the Gram-negative organisms (FIG. 13A). In some instances, gene expression differed mainly in the magnitude of the response; Gram-negative organisms induced quantitatively higher expression levels of these genes than did the Gram-positive organisms. In other instances, gene expression changes were qualitatively different between these two classes of organisms; Gram-negative bacteria modulated gene expression and the Gram-positive organisms did not (FIG. 13A, pink bar). The expression of six genes were repressed by Gram-negative organisms (FIG. 13B, left). Another nine genes appeared to be induced by Gram-positive organisms and were unchanged or repressed by Gram-negative organisms (FIGS. 1 and 14A, left panel).

Lipopolysaccharide (LPS) is present in the outer membrane of Gram-negative organisms and distinguishes the two groups of bacteria used in these studies. LPS is a TLR4 agonist, raising the possibility that the pattern of gene expression that differentiated Gram-negative and Gram-positive organisms was due to signaling by TLR4. This possibility was tested by measuring the responses of macrophages treated with LPS and other bacterial components. LPS recapitulated the expression pattern of the 101 genes that were differentially regulated by whole Gram-negative bacteria (FIG. 13B, right panel; Tables 2, 3 and 4, GenBank accession numbers and common names are listed). In particular, expression changes of the genes that best discriminated between macrophages exposed to Gram-negative and Gram-positive organisms were reproduced by LPS, defining a TLR4-induced core cluster of genes. In contrast, muramyl dipeptide (MDP) and lipoteichoic acid (LTA) induced gene expression changes similar to the Gram-positive organisms. These two components, however, failed to induce the cluster of nine genes that appeared specific to exposure to Gram-positive bacteria (FIG. 13B). A non-TLR agonist, formyl-MetLeuPhe, failed to induce expression changes that were characteristic of either class of bacteria. Together, these results identify LPS and TLR4 as molecules that are important for the differential regulation of gene expression by Gram-negative bacteria.

TABLE 2
Differentially expressed genes in macrophages
Genes induced by Gram negative bacteria

| | | | |
|---|---|---|---|
| X66867_CDS1_AT | MAX | X63717_AT | TNFRSF6 |
| M80899_AT | AHNAK | U65416_RNA1_S_AT | MICB |
| M27492_AT | IL1R1 | M87434_AT | OAS2 |
| X04327_AT | BPGM | M62403_S_AT | IGFBP4 |
| M34458_RNA1_S_AT | LMNB1 | U34877_AT | BLVRA |
| U26173_S_AT | NFIL3 | M59830_AT | HSPA1B |
| X83492_S_AT | X83492 | HG4297-HT4567_AT | HG4297-HT4567 |
| M68520_AT | CDK2 | D86979_AT | KIAA0226* |
| U50062_AT | RIPK1 | D43949_AT | KIAA0082* |
| M24470_AT | GMPR | X78711_AT | GK |
| M13690_S_AT | C1NH | U55766_AT | HRB2 |
| M88163_AT | SMARCA1 | U56998_AT | CNK |
| L37792_AT | STX1A | X54489_RNA1_AT | GRO1 |
| X02530_AT | SCYB10 | X68277_AT | DUSP1 |
| U50527_S_AT | U50527 | D14874_AT | ADM |
| U00672_AT | IL10RA | M58603_AT | NFKB1 |
| S79639_AT | EXT1 | M31627_AT | XBP1 |
| M92843_S_AT | ZFP36 | L15326_S_AT | PTGS2 |
| D90070_S_AT | PMAIP1 | L09229_S_AT | FACL1 |
| X83490_S_AT | X83490 | L40377_AT | PI8 |
| U59286_AT | SCYB11 | L00352_AT | LDLR |
| M87284_AT | OAS2 | U77180_AT | SCYA19 |
| M30818_AT | MX2 | U01824_AT | U01824 |
| M14660_AT | LFIT2 | M27533_S_AT | CD80 |
| L40387_AT | OASL | M33684_S_AT | PTPN1 |
| X99886_S_AT | SCYA8 | L38608_AT | ALCAM |
| HG3417-HT3600_S_AT | HG3417-HT3600 | HG2981- | HG2981-HT3938 |
| | | HT3938_S_AT | |
| U50648_S_AT | PRKR | U88964_AT | ISG20 |
| M33882_AT | MX1 | HG3415-HT3598_AT | HG3415-HT3598 |
| M13755_AT | ISG15* | M30894_AT | TRG@ |
| U52513_AT | IFIT4 | L08069_AT | HSJ2 |
| M24594_AT | IFIT1 | M34455_AT | INDO |
| D14889_AT | RAB33A | D84276_AT | CD38 |
| X57351_S_AT | IFITM2 | U31628_AT | IL15RA |
| U22970_RNA1_S_AT | GIP3 | HG1612-HT1612_AT | HG1612-HT1612 |
| X67325_AT | IFI27 | M55543_AT | GBP2 |
| J04164_AT | IFITM1 | L31584_AT | CCR7 |
| X57351_AT | IFITM2 | AF005775_AT | CFLAR |
| X99699_AT | HSXIAPAF1* | D30755_AT | D30755 |
| U10439_AT | ADAR | | |
| X72755_AT | MIG | | |
| X02875_S_AT | OAS1 | | |
| D28915_AT | MTAP44* | | |
| D13146_CDS1_AT | CNP | | |
| AB000115_AT | GS3686* | | |
| L29277_AT | STAT3 | | |
| L22342_AT | IFI41 | | |

TABLE 3
Genes repressed by Gram-negative bacteria

| | |
|---|---|
| Z11559_AT | ACO1 |
| X62055_AT | PTPN6 |
| U62293_RNA1_S_A | LIMK1 |
| T | |
| S82447_S_AT | GCN5L1 |
| J03589_AT | UBL4 |
| D50917_AT | KIAA0127* |

TABLE 4
Genes induced by Gram-positive bacteria

| | |
|---|---|
| U75370_AT | POLRMT |
| L04270_AT | LTBR |
| HG4334-HT4604_S_AT | HG4334-HT4604 |
| X05610_AT | COL4A2 |
| HG1686-HT4572_S_AT | HG1686-HT4572 |
| U90902_AT | U90902 |
| AB000584_AT | PLAB* |
| M91083_AT | C11ORF13 |
| L10910_AT | CC1.3* |

Regulation of Gene Expression by IFN

Hierarchical clustering of the differentially-regulated genes highlighted the TLR4-induced core cluster of genes (pink bar, FIGS. 13A and 13B). This cluster contained genes most strongly induced by Gram-negative organisms and LPS that were most different between the two classes of bacteria. Twenty-six of the 43 genes in this cluster are known to be regulated by IFNs (FIG. 14, left panel). This association, coupled with the fact that many genes of this cluster increased 6 hours into the time course, suggested these genes were secondarily regulated by IFNs induced by Gram-negative bacteria.

The question of whether IFNs were sufficient to elicit the gene expression changes that were observed after macrophages were exposed to Gram-negative bacteria was addressed. Expression profiles of macrophages incubated with IFN-α, -β, or -γ demonstrated the IFNs were sufficient to induce the expression of the TLR4-induced core cluster of genes and the majority of changes seen in the other genes (FIG. 14, right panel). In contrast, IL-10 and IL-12 failed to induce comparable gene expression changes. These results implicated IFNs in the macrophage gene expression profile associated with Gram-negative bacteria and LPS.

IFN-Dependent Production of 1-TAC after TLR4 Activation

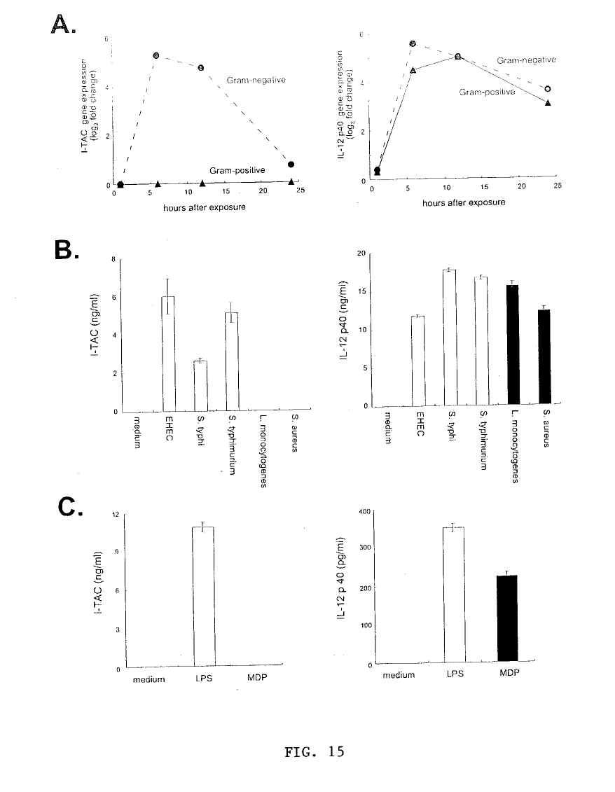

To confirm the findings of the array experiments and to investigate the mechanism of gene regulation, one member of the TLR4-induced core cluster was selected as a marker for expression changes specific to Gram-negative bacteria. CXCL11, also known as I-TAC, is a chemokine that is known to be induced by IFN (Cole, K. et al., 1998. J. Exp. Med., 187:2009-2021). I-TAC gene expression was closely correlated with exposure of human macrophages to Gram-negative bacteria (FIG. 15A). Both Gram-positive and Gram-negative bacteria induced IL-12 p40 gene expression (FIG. 15A). Supernatants of macrophages exposed to Gram-negative organisms contained readily-detectable levels of I-TAC (FIG. 15B). In contrast, Gram-positive organisms failed to elicit I-TAC secretion even though both classes of organisms induced IL-12 p40 (FIG. 15B). Consistent with the idea that TLR4 is stimulated by the Gram-negative bacteria, I-TAC production was induced by LPS but not by MDP (FIG. 15C) or LTA. However, both LPS and MDP elicited IL-12 p40 secretion (FIG. 15C).

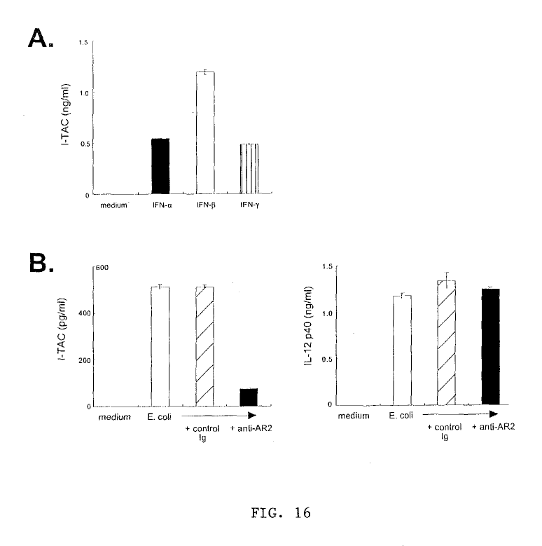

Consistent with the array data, it was found that IFN-α, -β, and -γ were sufficient to induce I-TAC production (FIG. 16A). A monoclonal antibody that blocks the Type I IFN receptor significantly reduced I-TAC production induced by E. coli (FIG. 16B). The production of IL-12 p40 was not affected by neutralization of IFNAR2 (FIG. 16B). Phosphorylation of macrophage STAT1 in response to LPS was also observed, consistent with the activation of type I IFN receptors after TLR4 activation. While not wishing to be bound by theory, these findings are consistent with the model that IFN-β contributes to the differential responses induced by TLR4 stimulation in murine macrophages and human dendritic cells (Toshchakov, V. et al., 2002. Nat. Immunol., 3:392-398; Re, F. and Strominger, J., 2001. J. Biol. Chem., 276:37692-37699; Fujihara, M. et al., 1994. J. Biol. Chem., 269:12773-12778; Doyle, S. et al., 2002. Immunity, 17:251-263).

IFN-Independent Production of IL-12 p70 after TLR4 Activation

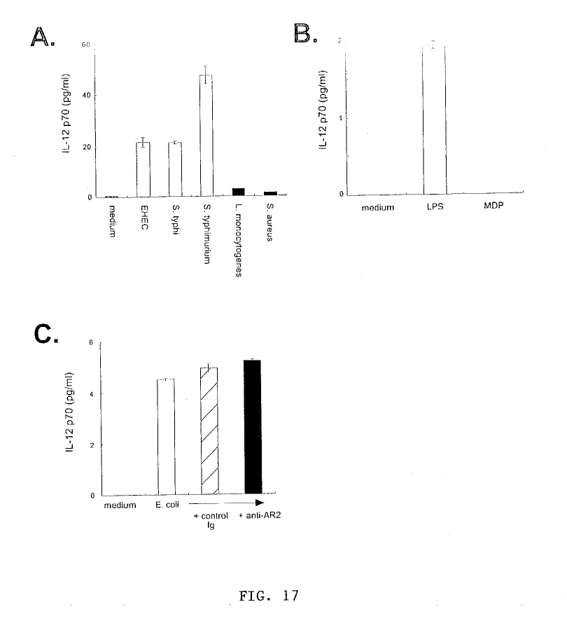

The p35 component of bioactive IL-12 p70 was not present in the cluster of genes associated with stimulation by Gram-negative organisms even though it has been associated with LPS stimulation of TLR4 (Re, F. and Strominger, J., 2001. J. Biol. Chem., 276:37692-37699; Ma, X. et al., 1996. J. Exp. Med., 183:147-157; Salkowski, C. et al., 1999. J. Immunol., 163:1529-1536; Hirschfeld, M. et al., 2001. Infect. Immun., 69:1477-1482). Similar to the observations of Re and Strominger, inspection of the microarray data showed p35 gene expression was low and changes were detected in only two of the eight time courses in which macrophages were exposed to Gram-negative bacteria. As a result, p35 failed to meet the cutoff criteria for statistical significance in the analysis. It was reasoned that p70 protein in the supernatants from macrophages exposed to Gram-negative and Gram-positive bacteria might be a more reliable, alternative assessment of p35 expression. By ELISA of culture supernatants, macrophages exposed to Gram-negative organisms produced substantially more p70 protein than those exposed to Gram-positive organisms (FIG. 17A). LPS also stimulated p70 production although MDP did not (FIG. 17B). In contrast to I-TAC, however, IFNs were insufficient to induce p70 production. Moreover, the anti-IFNAR2 monoclonal antibody that neutralizes the type I IFN receptor failed to block production of IL-12 p70 by macrophages exposed to E. coli (FIG. 17C), identifying an IFN-independent mechanism of gene expression changes after TLR4 activation.

IL-12 p70 Production by Mixed Cell Populations

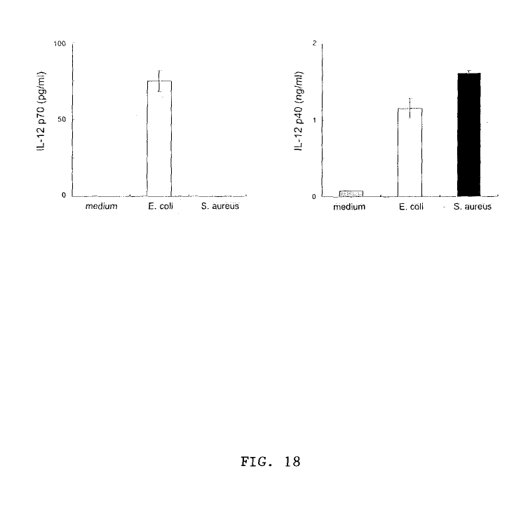

Because I-TAC and IL-12 p70 were produced primarily after macrophages encountered Gram-negative bacteria, these cytokines might be used to discriminate between infections caused by Gram-negative and Gram-positive bacteria. However, a bacterium is likely to encounter a mixed population of inflammatory cells during an infection. Therefore, the question of whether a mixed population of host cells would specifically produce I-TAC and IL-12 p70 after TLR4 activation was addressed. One source of inflammatory cells is unfractionated peripheral blood, which contains monocytes and neutrophils that bear TLR. When the cellular components of whole blood were incubated with E. coli, IL-12 p70 accumulated in the supernatants (FIG. 18). In contrast, S. aureus failed to induce p70. Both bacteria induced the production of IL-12 p40 in the supernatants (FIG. 18). I-TAC, however, was not detectable in the supernatants of whole blood cells incubated with either bacterium (not shown). Thus, a mixed population of cells is faithful to the specific production of IL-12 p70 after exposure to E. coli, similar to purified macrophages.

Discussion

Understanding the innate immune response to infections is critical to understanding inflammation and induction of adaptive immunity. Studies of the innate immune response to purified bacterial components have identified TLR-specific signals and gene expression changes. Yet, it is crucial to understand how the innate immune system responds to whole organisms that cause infections.

The results described herein demonstrate the innate immune response to whole bacteria is a consequence of the cumulative activation of TLR, as proposed by Underhill and Ozinsky (Underhill, D. and Ozinsky, A., 2002. Curr. Opin. Immunol., 14:103-110). Gram-negative bacteria activate gene expression changes that encompass those induced by Gram-positive bacteria, defined as the macrophage activation program. It is shown herein that Gram-negative bacteria induce an additional set of genes through IFN-dependent and IFN-independent mechanisms. The differences in expression profiles induced by the two classes of bacteria are largely attributable to LPS, consistent with a specific role for TLR4. Thus, the differential response to the class of Gram-negative bacteria can be ascribed to the activation of an additional TLR.

Several lines of evidence argue that the Gram-negative-specific expression profile is activated by TLR4. The gene expression changes induced by Gram-negative organisms were also induced by E. coli LPS, a known agonist for TLR4. The TLR4 core cluster contains IP-10 (CXCL10, FIG. 14) and GARG16 (IFIT1, FIG. 14), genes that have been identified in other studies of LPS activation of macrophages and dendritic cells (Kawai, T. et al., 2001. J. Immunol., 167:5887-5894; Toshchakov, V. et al., 2002. Nat. Immunol., 3:392-398; Re, F. and Strominger, J., 2001. J. Biol. Chem., 276:37692-37699). I-TAC production, a marker for the expression changes induced by Gram-negative organisms, is dependent on an IFN autocrine feedback loop similar to that observed in murine macrophages stimulated with LPS.

Identified herein is an alternative mechanism by which TLR4 regulates differential gene expression. Toshchakov and colleagues have recently demonstrated an IFN-dependent pathway that leads to specific gene expression changes after TLR4 stimulation (Toshchakov, V. et al., 2002. Nat. Immunol., 3:392-398). Consistent with this mechanism, the majority of the expression profile observed after TLR4 activation was recapitulated by IFNs and Type I IFN was necessary and sufficient for I-TAC production. In contrast, the data described herein show that IL-12 p70 was produced in response to Gram-negative bacteria and E. coli LPS, but the regulation of this cytokine was different than that of I-TAC. IFNs were not sufficient and IFNAR2 was not necessary for IL-12 p70 production after TLR4 stimulation. Thus, IL-12 p70 production after TLR4 activation appears to be independent of IFN.

Although LPS recapitulated the majority of the response induced by Gram-negative bacteria, the gene expression changes induced by LPS were less robust in magnitude and duration. This may be related to differences between free LPS and LPS presented on the surface of whole bacteria. Whole Gram-negative bacteria may deliver quantitatively more LPS to the macrophage. However, it is also likely that whole Gram-negative bacteria synergistically activate multiple TLR compared to purified LPS, thereby eliciting a more potent inflammatory response (Sato, S. et al., 2000. J. Immunol., 165:7096-7101).

While a robust TLR4-specific pattern of expression changes was observed, the analysis identified few genes that were induced uniquely by Gram-positive bacteria. It was possible that Gram-positive organisms would generate a specific pattern of expression changes because IL-8 has been specifically associated with TLR2 signaling in human monocyte-derived dendritic cells. In the datasets described herein, however, IL-8 is a gene in the macrophage activation program that is induced by diverse bacteria. Because whole bacteria possess molecules like peptidoglycan that activate TLR2, IL-8 is not likely to discriminate between the different classes of bacteria studied here. The few expression changes that were induced by Gram-positive bacteria were of low magnitude. Nevertheless, the TLR2-specific agonists MDP and LTA were insufficient to induce this small cluster of genes, suggesting these expression changes are induced by some other molecule(s) in Gram-positive bacteria.

The data described herein demonstrate that Gram-negative bacteria are more potent than Gram-positive bacteria at inducing IL-12 p70. While others have shown that S. aureus can induce this critical regulator of adaptive immune responses (D'Andrea, A. et al., 1992. J. Exp. Med., 176:1387-1398; Cousens, L. et al., 1997. Proc. Natl. Acad. Sci. USA, 94:634-639; Ma, X. et al., 2000. J. Immunol., 164:1722-1729), little or no p70 production by human macrophages stimulated by Gram-positive organisms and bacterial components that activate TLR2 was observed herein. Titrating the S. aureus multiplicity of infection over five orders of magnitude and using the same strain of fixed S. aureus (Pansorbin, Calbiochem) employed by previous investigators (D'Andrea, A. et al., 1992. J. Exp. Med., 176:1387-1398; Cousens, L. et al., 1997. Proc. Natl. Acad. Sci. USA, 94:634-639; Ma, X. et al., 2000. J. Immunol., 164:1722-1729) failed to enhance p70 production. While S. aureus and L. monocytogenes can induce low levels of this cytokine (FIG. 17A), the production of p70 appears most robust after TLR4 activation. IL-12 p70 is an important modulator of host defenses. It remains to be determined how differential production of cytokines, including IL-12 p70 and I-TAC, influences the host inflammatory and adaptive immune responses to infection by Gram-negative organisms.

Differential production of IL-112 p70 by cells in whole blood suggests a means to discriminate between clinical infections caused by Gram-negative and Gram-positive bacteria. In the case of I-TAC, several IFNs induce I-TAC expression, which may limit its specificity for Gram-negative infections. In addition, molecules like I-TAC might be consumed shortly after production which could account for the failure to detect I-TAC in the supernatants from whole blood cultured with E. coli. Further investigation of expression profiles from in vitro infections and ex vivo from patients should help to identify additional candidate diagnostic markers.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying infection by a pathogen comprising the steps of:
a) isolating mRNA from a whole blood sample; and
b) determining gene expression of at least one stimulus-specific gene,
wherein expression of a stimulus-specific gene is indicative of infection by a pathogen to which the stimulus-specific gene is specific.

2. The method of claim 1, wherein stimulus-specific gene expression is increased.

3. The method of claim 2, wherein the stimulus-specific gene is selected from the group consisting of: X66867_CDSI_AT, M80899_AT, M27492_AT, X04327_AT, M34458_RNA1_S_AT, U26173_S_AT, X83492_S_AT, M68520_AT, U50062_AT, M24470_AT, M13690 S_AT, M88163_AT, L37792_AT, X02530_AT, U50527_S_AT, U00672_AT, S79639_AT, M92843_S_AT, D90070_S_AT, X83490_S_AT, U59286_AT, M87284_AT, M30818_AT, M14660_AT, L40387 AT, X99886_S_AT, HG3417-HT3600_S_AT, U50648_S_AT, M33882_AT, M13755_AT, U52513_AT, M24594_AT, D14889_AT, X57351_S_AT, U22970_RNA1_S_AT, X67325_AT, J04164_AT, X57351_AT, X99699_AT, U10439_AT, X72755_AT, X02875_S_AT, D28915_AT, D13146_CDS1_AT, AB00015_AT, L29277_AT, L22342_AT, X63717_AT, U65416_RNA1_S_AT, M87434_AT, M62403_S_AT, U34877_AT, M59830_AT, HG4297-HT4567_AT, D86979_AT, D43949_AT, X78711_AT, U55766_AT, U56998_AT, X54489_RNA1_AT, X68277_AT, D14874_AT, M58603_AT, M31627_AT, L15326_S_AT, L09229_S_AT, L40377_AT, L00352_AT, U77180_AT, U01824_AT, M27533 S_AT, M33684_S_AT, L38608_AT, HG2981-HT3938_S_AT, U88964_AT, HG3415-HT3598_AT, M30894_AT, L08069_AT, M34455_AT, D84276_AT, U31628 AT, HG1612-HT1612_AT, M55543_AT, L31584_AT, AF005775_AT and D30755_AT.

4. The method of claim 2, wherein the stimulus-specific gene is selected from the group consisting of: I-TAC, IL-12 p35 and IL-12 p40.

5. The method of claim 2, wherein the stimulus-specific gene is selected from the group consisting of: U75370_AT, L04270_AT, HG4334-HT4604_S_AT, X05610_AT, HG1686-HT4572_S_AT, U90902_AT, AB000584_AT, M91083_AT and L10910_AT.

6. The method of claim 1, wherein stimulus-specific gene expression is decreased.

7. The method of claim 6, wherein the stimulus-specific gene is selected from the group consisting of: Z11559_AT, X62055_AT, U62293_RNA1_S_AT, S82447_S_AT, J03589_AT and D50917_AT.

8. The method of claim 1, wherein at least one stimulus-specific gene is not expressed.

9. The method of claim 1, wherein the sample is an isolated monocyte sample derived from whole blood.

10. The method of claim 9, wherein the isolated monocyte sample is differentiated in culture into macrophages.

11. A method of classifying a pathogen comprising the steps of:
a) exposing macrophages with a pathogen or immunogenic components thereof;
b) isolating and labeling mRNA from said macrophages;
c) detecting labeled mRNA from said macrophages such that a gene expression profile is produced; and
d) analyzing the gene expression profile relative to one or more reference gene expression profile(s),
such that similarities between the gene expression profile of the pathogen-exposed macrophage and at least one reference gene expression profile classify the pathogen as belonging to the class corresponding to the reference gene expression profile.

12. A method of diagnosing infection in a mammal comprising the steps of:
a) isolating mRNA from a whole blood sample obtained from the mammal; and
b) contacting the mRNA with at least one stimulus-responsive gene probe wherein hybridization of a stimulus-responsive probe to the mRNA is indicative of infection in said mammal.

13. The method of claim 12, wherein the stimulus-responsive gene is selected from the group consisting of: GCSF, GMCSF, IL12B, IL1RN, IL6, IL6, PBEP, ProIL1B, TNFA, IL8, IL8, IP10, MCP1, MGSA, MIP1A, MIPIB, MIP2A, MIP2B, RANTES, CD44, CD44, ICAM1, IFITM1, LAMB3, NINJ1, TNFA1P6, ADORA2A, CCR6, CCR7, CCRL2, DTR, EBI3, HM74, ILISRA, IL7R, LDLR, P2RX7, P2XR, PLAUR, PVR, SLAM, TNFRSF5, TXN, CNK, DUSP1, DUSP2, DUSP5, EBI2, GBP1, HCK, INHBA, JAG1, KYNU, LIMK2, MAP2K3, MAP3K4, MINOR, NAF1, NFKB1, PDE4B, PPP3CC, PTPN1, TRAF1, TRIP10, DSCR1, ELF4, ETS2, IRF1, IRLB, JUNB, MRF-1, NFKBIA, NFKBIE, NFKBp50, STAT4, STAT5A, TSC22, XBP1, ZFP36, COX2, COX2, COX2, GCH1, PTX3, BIRC2, BIRC3, BIRC3, CFLAR, IER3, TNFAIP3, BTG1, BTG3, TNFRSF9, TNFSF9, DAP, MMP1, MMP10, MMP14, SERPINB2, SERPINB8, GADD45A, HSPA1A, SOD2, SOD2, ATP2B1, NRAMP2, SLC7A5, ADA, AMPD3, beta-1,4-galatosyl transferase, BF, CKB, GJB2, GLCLR, HSD11B1, INDO, MTF1, ADM, ARHH, B4-2, BRCA2, CD83, GEM, GOS, GYPC, H2AFO, HIVEP2, ISG15, ISG20, MACMARKS, MIG2, MXJ, RCN1, SDC4, SNL, TNFAIP2, TSSC3, K1AA0105, KIAA0172, GCHFR, CAT, LTA4H, CD14, ALOX5, MCP1, PECAM1, SPARC, RARA, TNFRSF1A, ENG, L77730, CD36L1, CD163, TGFBR2, GSF1R, MRC1, CD32, P2RX1, SRPK2, SLA, MERTK, DAB2, SF3A3, EGR2, NFATC3, MX11, FOS, Hbrm, SLC29A1, UPA, FGL2, TBXAS1, SGSH, PPP2R5C, IDH2, LPL, MPI, PYGL, HSD17B4, RNASE6, GALC, GLCLC, RNASE1, ME1, PURA, MYO1E, VCL, IV12B, ADFP, MNDA, STAB1, TGFBI, KIAA0022, AD000092, D87075, U79288, P311, HG2090-HT2090, HG2090-HT2090 and HG2090-HT2090.

14. The method of claim 12, wherein the stimulus-responsive gene probe is a stimulus-specific gene probe.

15. The method of claim 12, wherein the stimulus-responsive gene probe is a common stimulus-responsive gene probe.

16. A method of diagnosing infection by a pathogen in a mammal comprising the steps of:
a) isolating mRNA from a whole blood sample obtained from the mammal; and
b) determining gene expression of at least one stimulus-specific gene,
wherein expression of the stimulus-specific gene is indicative of infection by a pathogen to which the stimulus-specific gene is specific.

17. The method of claim 16, wherein the stimulus-specific gene is selected from the group consisting of: X66867_CDS1_AT, M80899_AT, M27492_AT, X04327_AT, M34458_RNA1_S_AT, U26173_S_AT, X83492_S_AT, M68520_AT, U50062_AT, M24470_AT, M13690_S_AT, M88163_AT, L37792 AT, X02530_AT, U50527_S_AT, U00672_AT, S79639_AT, M92843_S_AT, D90070_S_AT, X83490_S_AT, U59286_AT, M87284_AT, M30818_-AT, M14660_AT, L40387_AT, X99886_S_AT, HG3417-HT3600_S_AT, U50648_S_AT, M33882_AT, M13755_AT, U52513_AT, M24594 AT, D14889_AT, X57351_S_AT, U22970_RNA1_S_AT, X67325_AT, J04164 AT, X57351 AT, X99699 AT, U10439 AT, X72755_AT, X02875_S_AT, D28915_AT, D13146_CDS1AT, AB00015_AT, L29277_AT, L22342_AT, X63717_AT, U65416_RNA1_S_AT, M87434_AT, M62403_S_AT, U34877 AT, M59830_AT, HG4297-HT4567_AT, D86979_AT, D43949_AT, X78711 AT, U55766_AT, U56998_AT, X54489_RNA1_AT, X68277_AT, D14874_AT, M58603_AT, M31627_AT, L15326_S_AT, L09229_S_AT, L40377_AT, L00352_AT, U77180_AT, U01824_AT, M27533_S_AT, M33684_S_AT, L38608_AT, HG2981-HT3938_S_AT, U88964_AT, HG3415-HT3598_AT, M30894_AT, L08069_AT, M34455_AT, D84276_AT, U31628_AT, HG1612-HT1612_AT, M55543_AT, L31584_AT, AF005775_AT and D30755_AT.

18. The method of claim 16, wherein the stimulus-specific gene is selected from the group consisting of: I-TAC, IL-12 p35 and 1L-12 p40.

19. The method of claim 16, wherein the stimulus-specific gene is selected from the group consisting of: U75370_AT, L04270_AT, HG4334-HT4604_S_AT, X05610_AT, HG1686-HT4572_S_AT, U90902_AT, AB000584_AT, M91083_AT and L10910_AT.

20. The method of claim 16, wherein stimulus-specific gene expression is increased.

21. The method of claim 16, wherein stimulus-specific gene expression is decreased.

22. A method of formulating a therapeutic regimen for treating a pathogenic infection comprising the steps of:
a) identifying a pathogen that causes the infection from a diagnostic assay performed on a whole blood sample; and
b) formulating the therapeutic regimen according to the pathogen identified.

23. The method of claim 22, comprising repeating steps (a) and (b).

24. A method of optimizing a vaccine comprising the steps of:
a) contacting one or more macrophages with at least one test vaccine;
b) isolating mRNA from said macrophages;
c) determining gene expression profiles in said macrophages; and
e) comparing the macrophage gene expression profile to a reference gene expression profile,
wherein a similarity to a pathogen-specific or pathogen-responsive gene expression profile is indicative of an optimized vaccine.

25. An ex vivo therapeutic treatment for a disorder selected from the group consisting of pathogenic infection, sepsis and autoimmunity comprising the steps of:
a) contacting a patient's macrophages with a pathogen or components thereof, such that the macrophages would normally signal an immune response, thereby producing activated macrophages;
b) returning the activated macrophages to the patient such that the activated macrophages trigger an immune response against the pathogen.

26. A method of measuring the immune response to a stimulus comprising the steps of:
a) contacting a whole blood sample with a stimulus;
b) isolating mRNA from the sample; and
c) determining a gene expression profile such that the expression of at least one stimulus-responsive gene measured,
thereby indicating the level of the immune response.

27. A method of measuring the immune response to a stimulus comprising the steps of:
a) contacting macrophages with a stimulus;
b) isolating and labeling mRNA from the macrophages;
c) contacting a DNA microarray with labeled mRNA from the macrophages; and
d) measuring and analyzing the gene expression profile relative to control stimulus such that at least one stimulus-responsive gene is identified which is indicative of an immune response.

28. The method of claim 27, wherein the macrophages obtained by culturing monocytes.

29. The method of claim 27, wherein the stimulus is selected from the group consisting of bacteria, fungi, viruses, or components thereof.

30. The method of claim 27, wherein the stimulus is selected from the group consisting of Escherichia coli, enterohemorrhagic E. coli O157:H7 (EHEC), Salmonella typhi, Salmonella typhimurium, Staphylococcus aureus, Listeria monocytogenes, M. tuberculosis, Mycobacterium bovis bacille Calmette-Guérin (BCG), lipopolysaccharide (LPS), polyI:C, and yeast mannan.

31. The method of claim 27, wherein the DNA microarray is Affymetrix HU 6800.

32. The method of claim 27, wherein the expression of the stimulus-responsive gene is increased in response to the stimulus.

33. The method of claim 27, wherein the expression of the stimulus-responsive gene is decreased in response to the stimulus.

34. The method of claim 27, wherein the stimulus-responsive gene is stimulus-specific.

35. A method of measuring the gene expression profile in macrophages in response to a stimulus comprising the steps of:
a) contacting immature macrophages with a stimulus;
b) isolating and labeling mRNA from the macrophages;
c) contacting a DNA microarray with labeled mRNA from the macrophages; and
d) measuring and analyzing the gene expression profile relative to control stimulus such that at least one stimulus-responsive gene is identified.

36. The method of claim 35, wherein the macrophages obtained by culturing monocytes.

37. The method of claim 35, wherein the stimulus is selected from the group consisting of bacteria, fungi, viruses, or components thereof.

38. The method of claim 35, wherein the stimulus is selected from the group consisting of Escherichia coli, enterohemorrhagic E. coli O157:H7 (EHEC), Salmonella typhi, Salmonella typhimurium, Staphylococcus aureus, Listeria monocytogenes, M. tuberculosis, Mycobacterium bovis bacille Calmette-Guérin (BCG), lipopolysaccharide (LPS), polyI:C, and yeast mannan.

39. The method of claim 35, wherein the DNA microarray is Affymetrix HU 6800.

40. The method of claim 35, wherein the expression of the stimulus-responsive gene is increased in response to the stimulus.

41. The method of claim 35, wherein the expression of the stimulus-responsive gene is decreased in response to the stimulus.

42. The method of claim 35, wherein the stimulus-responsive gene is stimulus-specific.

43. A method for generating a database of stimulus-responsive genes comprising the steps of:
a) contacting macrophages with a stimulus;
b) isolating and labeling mRNA from the macrophages;
c) contacting a DNA microarray with labeled mRNA from the macrophages; and
d) measuring and analyzing the gene expression profile relative to a control stimulus such that a database comprising at least one stimulus-responsive gene is generated.

44. A method of generating a database of stimulus-specific genes comprising the steps of:
a) contacting immature macrophages with a stimulus;
b) isolating and labeling mRNA from the macrophages;
c) contacting a DNA microarray with labeled mRNA from the macrophages; and
d) measuring and analyzing the gene expression profile relative to control stimulus such that a database of stimulus-specific genes comprising at least one stimulus-specific gene is generated.

45. A method of generating a database of common stimulus-responsive genes comprising the steps of:
a) contacting macrophages with a stimulus;
b) isolating and labeling mRNA from the macrophages;
c) contacting a DNA microarray with labeled mRNA from the macrophages; and
d) measuring and analyzing the gene expression profile relative to control stimulus such that a database of common stimulus-responsive genes comprising at least one common stimulus-responsive gene is generated

46. A database of stimulus-responsive genes.

47. The database of claim 46, comprising one or more genes selected from the group consisting of: GCSF, GMCSF, IL12B, IL1RN, IL6, IL6, PBEP, ProIL1B, TNFA, IL8, IL8, IP10, MCP1, MGSA, MIP1A, MIPIB, MIP2A, MIP2B, RANTES, CD44, CD44, ICAM1, IFITM1, LAMB3, NINJ1, TNFA1P6, ADORA2A, CCR6, CCR7, CCRL2, DTR, EBI3, HM74, ILI5RA, IL7R, LDLR, P2RX7, P2XR, PLAUR, PVR, SLAM, TNFRSF5, TXN, CNK, DUSP1, DUSP2, DUSP5, EBI2, GBP1, HCK, INHBA, JAG1, KYNU, LIMK2, MAP2K3, MAP3K4, MINOR, NAF1, NFKB1, PDE4B, PPP3CC, PTPN1, TRAF1, TRIP10, DSCR1, ELF4, ETS2, IRF1, IRLB, JUNB, MRF-1, NFKBIA, NFKBIE, NFKBp50, STAT4, STAT5A, TSC22, XBP1, ZFP36, COX2, COX2, COX2, GCH1, PTX3, BIRC2, BIRC3, BIRC3, CFLAR, IER3, TNFAIP3, BTG1, BTG3, TNFRSF9, TNFSF9, DAP, MMP1, MMP10, MMP14, SERPINB2, SERPINB8, GADD45A, HSPA1A, SOD2, SOD2, ATP2B1, NRAMP2, SLC7A5, ADA, AMPD3, beta-1,4-galatosyl transferase, BF, CKB, GJB2, GLCLR, HSD11B1, INDO, MTF1, ADM, ARHH, B4-2, BRCA2, CD83, GEM, GOS, GYPC, H2AFO, HIVEP2, ISG15, ISG20, MACMARKS, MIG2, MXJ, RCN1, SDC4, SNL, TNFAIP2, TSSC3, K1AA0105, KIAA0172, GCHFR, CAT, LTA4H, CD14, ALOX5, MCP1, PECAM1, SPARC, RARA, TNFRSF1A, ENG, L77730, CD36L1, CD163, TGFBR2, GSF1R, MRC1, CD32, P2RX1, SRPK2, SLA, MERTK, DAB2, SF3A3, EGR2, NFATC3, MX11, FOS, Hbrm, SLC29A1, UPA, FGL2, TBXAS1, SGSH, PPP2R5C, IDH2, LPL, MPI, PYGL, HSD17B4, RNASE6, GALC, GLCLC, RNASE1, ME1, PURA, MYO1E, VCL, IV12B, ADFP, MNDA, STAB1, TGFBI, KIAA0022, AD000092, D87075, U79288, P311, HG2090-HT2090, HG2090-HT2090 and HG2090-HT2090.

48. A database of stimulus-specific genes.

49. The database of claim 48, wherein the stimulus-specific gene is selected from the group consisting of: X66867_CDS1_AT, M80899_AT, M27492_AT, X04327_AT, M34458_RNA1_S_AT, U26173_S_AT, X83492_S_AT, M68520 AT, U50062_AT, M24470_AT, M13690_S_AT, M88163_AT, L37792_AT, X02530_AT, U50527_S_AT, U00672_AT, S79639_AT, M92843_S_AT, D90070_S_AT, X83490_S_AT, U59286_AT, M87284_AT, M30818_AT, M14660_AT, L40387_AT, X99886_S_AT, HG3417-HT3600_S_AT, U50648_S_AT, M33882_AT, M13755_AT, U52513_AT, M24594_AT, D14889_AT, X57351_S_AT, U22970_RNA1_S_AT, X67325_AT, J04164_AT, X57351_AT, X99699_AT, U10439_AT, X72755_AT, X02875_S_AT, D28915_AT, D13146_CDS1_AT, AB00015_AT, L29277 AT, L22342_AT, X63717_AT, U65416_RNA1_S_AT, M87434 AT, M62403_S_AT, U34877_AT, M59830_AT, HG4297-HT4567_AT, D86979_AT, D43949_AT, X78711_AT, U55766_AT, U56998_AT, X54489_RNA1_AT, X68277_AT, D14874 AT, M58603_AT, M31627_AT, L15326_S_AT, L09229_S_AT, L40377 AT, L00352_AT, U77180 AT, U01824_AT, M27533_S_AT, M33684_S_AT, L38608_AT, HG2981-HT3938_S_AT, U88964_AT, HG3415-HT3598_AT, M30894 AT, L08069_AT, M34455_AT, D84276_AT, U31628_AT, HG1612-HT1612_AT, M55543_AT, L31584_AT, AF005775_AT and D30755_AT.

50. The database of claim 48, wherein the stimulus-specific gene is selected from the group consisting of: I-TAC, IL-12 p35 and IL-12 p40.

51. The database of claim 48, wherein the stimulus-specific gene is selected from the group consisting of: U75370_AT, L04270_AT, HG4334-HT4604_S_AT, X05610_AT, HG1686-HT4572_S_AT, U90902_AT, AB000584_AT, M91083_AT and L10910_AT.

52. The database of claim 48, wherein stimulus-specific gene expression is decreased.

53. The database of claim 52, wherein the stimulus-specific gene is selected from the group consisting of: Z11559_AT, X62055_AT, U62293_RNA1_S_AT, S82447_S_AT, J03589_AT and D50917_AT.

54. A database of common stimulus-responsive genes.

55. The database of claim 55 comprising one or more genes selected from the group consisting of: GCSF, GMCSF, IL12B, IL1RN, IL6, IL6, PBEP, ProIL1B, TNFA, IL8, IL8, IP10, MCP1, MGSA, MIP1A, MIPIB, MIP2A, MIP2B, RANTES, CD44, CD44, ICAM1, IFITM1, LAMB3, NINJ1, TNFA1P6, ADORA2A, CCR6, CCR7, CCRL2, DTR, EBI3, HM74, ILI5RA, IL7R, LDLR, P2RX7, P2XR, PLAUR, PVR, SLAM, TNFRSF5, TXN, CNK, DUSP1, DUSP2, DUSP5, EBI2, GBP1, HCK, INHBA, JAG1, KYNU, LIMK2, MAP2K3, MAP3K4, MINOR, NAF1, NFKB1, PDE4B, PPP3CC, PTPN1, TRAF1, TRIP10, DSCR1, ELF4, ETS2, IRF1, IRLB, JUNB, MRF-1, NFKBIA, NFKBIE, NFKBp50, STAT4, STAT5A, TSC22, XBP1, ZFP36, COX2, COX2, COX2, GCH1, PTX3, BIRC2, BIRC3, BIRC3, CFLAR, IER3, TNFAIP3, BTG1, BTG3, TNFRSF9, TNFSF9, DAP, MMP1, MMP10, MMP14, SERPINB2, SERPINB8, GADD45A, HSPA1A, SOD2, SOD2, ATP2B1, NRAMP2, SLC7A5, ADA, AMPD3, beta-1,4-galatosyl transferase, BF, CKB, GJB2, GLCLR, HSD11B1, INDO, MTF1, ADM, ARHH, B4-2, BRCA2, CD83, GEM, GOS, GYPC, H2AFO, HIVEP2, ISG15, ISG20, MACMARKS, MIG2, MXJ, RCN1, SDC4, SNL, TNFAIP2, TSSC3, K1AA0105, KIAA0172, GCHFR, CAT, LTA4H, CD14, ALOX5, MCP1, PECAM1, SPARC, RARA, TNFRSF1A, ENG, L77730, CD36L1, CD163, TGFBR2, GSF1R, MRC1, CD32, P2RX1, SRPK2, SLA, MERTK, DAB2, SF3A3, EGR2, NFATC3, MX11, FOS, Hbrm, SLC29A1, UPA, FGL2, TBXAS1, SGSH, PPP2R5C, IDH2, LPL, MPI, PYGL, HSD17B4, RNASE6, GALC, GLCLC, RNASE1, ME1, PURA, MYO1E, VCL, IV12B, ADFP, MNDA, STAB1, TGFBI, KIAA0022, AD000092, D87075, U79288, P311, HG2090-HT2090, HG2090-HT2090 and HG2090-HT2090.

56. A method of identifying a pathogen comprising the steps of:
a) contacting one or more immature macrophages with a stimulus;
b) isolating and labeling mRNA from said macrophages;
c) contacting a DNA microarray with labeled mRNA from said macrophages; and
d) measuring and analyzing the gene expression profile relative to control stimulus such that at least one stimulus-specific gene is identified thereby identifying a pathogen for which the stimulus-specific gene is specific.

57. The method of claim 56, wherein the stimulus-specific gene is selected from the group consisting of: X66867_CDS1_AT, M80899_AT, M27492_AT, X04327_AT, M34458_RNA1_S_AT, U26173_S_AT, X83492_S_AT, M68520_AT, U50062_AT, M24470 AT, M13690_S_AT, M88163_AT, L37792_AT, X02530_AT, U50527_S_AT, U00672_AT, S79639_AT, M92843_S_AT, D90070_S_AT, X83490_S_AT, U59286_AT, M87284_AT, M30818 AT, M14660_AT, L40387_AT, X99886_S_AT, HG3417-HT3600_S_AT, U50648_S_AT, M33882_AT, M13755_AT, U52513_AT, M24594_AT, D14889 AT, X57351_S_AT, U22970_RNA1_S_AT, X67325_AT, J04164_AT, X57351_AT, X99699_AT, U10439_AT, X72755_AT, X02875_S_AT, D28915_AT, D13146_CDS1_AT, AB00015_AT, L29277_AT, L22342_AT, X63717 AT, U65416_RNA1_S_AT, M87434_AT, M62403_S_AT, U34877_AT, M59830_AT, HG4297-HT4567_AT, D86979_AT, D43949_AT, X78711 AT, U55766_AT, U56998_AT, X54489_RNA1_AT, X68277_AT, D14874_AT, M58603 AT, M31627_AT, L15326_S_AT, L09229_S_AT, L40377_AT, L00352_AT, U77180_AT, U01824 AT, M27533_S_AT, M33684_S_AT, L38608_AT, HG2981-HT3938_S_AT, U88964_AT, HG3415-HT3598_AT, M30894_AT, L08069_AT, M34455_AT, D84276_AT, U31628_AT, HG1612-HT1612_AT, M55543_AT, L31584_AT, AF005775_AT and D30755_AT.

58. The method of claim 56, wherein the stimulus-specific gene is selected from the group consisting of: I-TAC, IL-12 p35 and IL-12 p40.

59. The method of claim 56, wherein the stimulus-specific gene is selected from the group consisting of: U75370_AT, L04270_AT, HG4334-HT4604_S_AT, X05610_AT, HG1686-HT4572_S_AT, U90902_AT, AB000584_AT, M91083_AT and L10910_AT.

60. The method of claim 56, wherein stimulus-specific gene expression is decreased.

61. The method of claim 60, wherein the stimulus-specific gene is selected from the group consisting of: Z11559_AT, X62055_AT, U62293_RNA1_S_AT, S82447_S_AT, J03589_AT and D50917_AT.

62. A method of diagnosing infection by a pathogen comprising the steps of:
a) isolating mRNA from a whole blood sample; and
b) determining a gene expression profile such that at least one stimulus-specific gene is identified, thereby identifying the pathogen.

63. A method of diagnosing infection by a pathogen comprising the steps of:
a) isolating and labeling mRNA from at least one whole blood sample from a mammal;
b) contacting a DNA microarray with labeled mRNA from the sample; and
c) measuring and analyzing the gene expression profile relative to control stimulus such that at least one stimulus-specific gene is identified thereby identifying the pathogen for which the stimulus-specific gene is specific.

64. A method of diagnosing infection in a mammal comprising the steps of:
a) isolating proteins from one or more samples from the mammal;
b) contacting the proteins with at least one stimulus-specific antibody, wherein binding of the stimulus-specific antibody to one or more proteins is indicative of infection in the mammal.

65. A gene expression profile comprising Gram-negative-specific genes.

66. The gene expression profile of claim 65 comprising expression of at least one gene selected from the group consisting of: X66867_CDS1_AT, M80899_AT, M27492_AT, X04327_AT, M34458 RNA1_S_AT, U26173_S_AT, X83492_S_AT, M68520 AT, U50062_AT, M24470_AT, M13690_S_AT, M88163_AT, L37792_AT, X02530_AT, U50527_S_AT, U00672_AT, S79639_AT, M92843_S_AT, D90070_S_AT, X83490_S_AT, U59286_AT, M87284_AT, M30818_AT, M14660_AT, L40387 AT, X99886_S_AT, HG3417-HT3600_S_AT, U50648_S_AT, M33882_AT, M13755_AT, U52513_AT, M24594_AT, D14889_AT, X57351_S_AT, U22970_RNA1_S_AT, X67325_AT, J04164_AT, X57351_AT, X99699_AT, U10439 AT, X72755_AT, X02875_S_AT, D28915_AT, D13146_CDS1_AT, AB00015_AT, L29277_AT, L22342_AT, X63717_AT, U65416_RNA1_S_AT, M87434_AT, M62403_S_AT, U34877 AT, M59830_AT, HG4297-HT4567_AT, D86979 AT, D43949_AT, X78711_AT, U55766_AT, U56998_AT, X54489_RNA1_AT, X68277_AT, D14874_AT, M58603_AT, M31627 AT, L15326_S_AT, L09229_S_AT, L40377_AT, L00352_AT, U77180_AT, U01824_AT, M27533_S_AT, M33684_S_AT, L38608_AT, HG2981-HT3938_S_AT, U88964_AT, HG3415-HT3598_AT, M30894_AT, L08069_AT, M34455_AT, D84276_AT, U31628_AT, HG1612-HT1612_AT, M55543_AT, L31584_AT, AF005775_AT, D30755_AT, Z11559 AT, X62055 AT, U62293_RNA1_S_AT, S82447_S_AT, J03589_AT and D50917_AT.

67. A gene expression profile comprising Gram-positive-specific genes.

68. The gene expression profile of claim 67 comprising expression of at least one gene selected from the group consisting of: U75370_AT, L04270_AT, HG4334-HT4604_S_AT, X05610_AT, HG1686-HT4572_S_AT, U90902_AT, AB000584_AT, M91083_AT and L10910_AT.

69. A method for screening for an agent that induces a pathogen-specific immune response comprising the steps of:
a) contacting at least one macrophage with a test agent;
b) isolating mRNA from the macrophage(s);
c) determining a gene expression profile from the isolate mRNA; and
d) comparing the gene expression profile obtained from the macrophages contacted by the test agent with a reference gene expression profile of an activated macrophage,
wherein a similarity in gene expression profiles indicates the test agent induces a pathogen-specific immune response.

70. A method of identifying a genus-specific response comprising the steps of:
a) isolating mRNA from a whole blood sample obtained from a mammal;
b) contacting a DNA microarray with labeled mRNA from said macrophages; and
c) comparing the gene expression profile to a reference genus-specific-induced gene expression profile,
wherein a similarity indicates a genus-specific response.

71. The method of claim 70, wherein the reference gene expression profile comprises expression values for IFN-β.
